SEQUENCE LISTING (45 sequences)

SEQ ID NO: 1 - protein, 21 aa, Artificial
  IFNgammaR2 heterologous signal peptide
MLPRLVVLLAAFLSRRLGSDA

SEQ ID NO: 2 - protein, 6 aa, Artificial
  linker peptide
GSGGGG

SEQ ID NO: 3 - protein, 8 aa, Artificial
  dimer peptide
GGCGTPGK

SEQ ID NO: 4 - protein, 454 aa, Artificial
  RhesusIL10Rl-RhesusFC
MLPRLVVLLAAFLSRRLGSDAHGTELPSPPSVWFEAEFFHHILHWTPIPNQSESTCYEVALLRYGTGRWNSISNCSQALSYDLTAVTLDLYRSNGYWARVRAVDGSRHSNWTVTNTRFSLDEVTLTVGSVKLEIHNGFILGKIQPPRPKMAPANDTYESIFRHFREYEIAIRKVPGNFTFTHKKVKHENFSLLTSGEVGEFCVQVKPSVTSRTNKGMWSKEECVSLTRQYFTVTNVGSGGGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRQQQGNVFSCSVMHEALHNHYTQKSLGGCGTPGK

SEQ ID NO: 5 - protein, 457 aa, Artificial
  IL10R1-HumanFC
MLPRLVVLLAAFLSRRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVGSGGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGGCGTPGK

SEQ ID NO: 6 - protein, 457 aa, Artificial
  RhIL10R1-RhFCh
MLPRLVVLLAAFLSRRLGSDAHGTELPSPPSVWFEAEFFHHILHWTPIPNQSESTCYEVALLRYGTGRWNSISNCSQALSYDLTAVTLDLYRSNGYWARVRAVDGSRHSNWTVTNTRFSLDEVTLTVGSVKLEIHNGFILGKIQPPRPKMAPANDTYESIFRHFREYEIAIRKVPGNFTFTHKKVKHENFSLLTSGEVGEFCVQVKPSVTSRTNKGMWSKEECVSLTRQYFTVTNVGSGGGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLTSKLTVDKSRQQQGNVFSCSVMHEALHNHYTQKSLSLSGGCGTPGK

SEQ ID NO: 7 - protein, 295 aa, Artificial
  Rhesus IL10R1-D3EVGBP
MLPRLVVLLAAFLSRRLGSDAHGTELPSPPSVWFEAEFFHHILHWTPIPNQSESTCYEVALLRYGTGRWNSISNCSQALSYDLTAVTLDLYRSNGYWARVRAVDGSRHSNWTVTNTRFSLDEVTLTVGSVKLEIHNGFILGKIQPPRPKMAPANDTYESIFRHFREYEIAIRKVPGNFTFTHKKVKHENFSLLTSGEVGEFCVQVKPSVTSRTNKGMWSKEECVSLTRQYFTVTNVGSGGGGYTCAIRSKEDVPNFKEKMTRVIKRKFNKQSHSYLTKFLGSTSNDITTFLSMLD

SEQ ID NO: 8 - protein, 295 aa, Artificial
  Human IL10R1-D3-EVGBP
MLPRLVVLLAAFLSRRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVGSGGGGYTCAIRSKEDVPNFKEKMTRVIKRKFNKQSHSYLTKFLGSTSNDITTFLSMLD

SEQ ID NO: 9 - protein, 508 aa, Artificial
  RhIL10R1-RhFCk-D3EVGBP
MLPRLVVLLAAFLSRRLGSDAHGTELPSPPSVWFEAEFFHHILHWTPIPNQSESTCYEVALLRYGTGRWNSISNCSQALSYDLTAVTLDLYRSNGYWARVRAVDGSRHSNWTVTNTRFSLDEVTLTVGSVKLEIHNGFILGKIQPPRPKMAPANDTYESIFRHFREYEIAIRKVPGNFTFTHKKVKHENFSLLTSGEVGEFCVQVKPSVTSRTNKGMWSKEECVSLTRQYFTVTNVGSGGGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSCEELTKNQVSLYCLVKGFYPSDIVVEWESSGQPENTYKTTPPVLDSDGSYFLYSKLTVDKSRQQQGNVFSCSVMHEALHNHYTQKSLSLSGSGGGGYTCAIRSKEDVPNFKEKMTRVIKRKFNKQSHSYLTKFLGSTSNDITTFLSMLD

SEQ ID NO: 10 - protein, 204 aa, Artificial
  Rhesus IgG1 FC domain
PSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRQQQGNVFSCSVMHEALHNHYTQKSL

SEQ ID NO: 11 - protein, 8 aa, Artificial
  dimer peptide
GGCGTPAS

SEQ ID NO: 12 - protein, 454 aa, Artificial
  RhIL-10R1-RhIgG1
MLPRLVVLLAAFLSRRLGSDAHGTELPSPPSVWFEAEFFHHILHWTPIPNQSESTCYEVALLRYGTGRWNSISNCSQALSYDLTAVTLDLYRSNGYWARVRAVDGSRHSNWTVTNTRFSLDEVTLTVGSVKLEIHNGFILGKIQPPRPKMAPANDTYESIFRHFREYEIAIRKVPGNFTFTHKKVKHENFSLLTSGEVGEFCVQVKPSVTSRTNKGMWSKEECVSLTRQYFTVTNVGSGGGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLGGCGTPGK

SEQ ID NO: 13 - protein, 290 aa, Artificial
  Rhehh10R1
MLPRLVVLLAAFLSRRLGSDAHGTELPSPPSVWFEAEFFHHILHWTPIPNQSESTCYEVALLRYGTGRWNSISNCSQALSYDLTAVTLDLYRSNGYWARVRAVDGSRHSNWTVTNTRFSLDEVTLTVGSVKLEIHNGFILGKIQPPRPKMAPANDTYESIFRHFREYEIAIRKVPGNFTFTHKKVKHENFSLLTSGEVGEFCVQVKPSVTSRTNKGMWSKEECVSLTRQYFTVTNVIIFFAFVLLLSGALAYCLALQLYVRRRKKLPRVLLFKKPNAFIFISQHPSPETQ

SEQ ID NO: 14 - protein, 300 aa, Artificial
  Rhxp10R1
MVAVILLATIPSLLLILGGWVLPAVVLTFFSPGTELPSPPSVWFEAEFFHHILHWTPIPNQSESTCYEVALLRYGTGRWNSISNCSQALSYDLTAVTLDLYRSNGYWARVRAVDGSRHSNWTVTNTRFSLDEVTLTVGSVKLEIHNGFILGKIQPPRPKMAPANDTYESIFSHFREYEIAIRKVPGNFTFTHKKVKHENFSLLTSGEVGEFCVQVKPSVTSRTNKGMWSKEECVSLTRQYFTVTNVIIFFAFVLLLSGALAYCLALQLYVRRRKKLPRVLLFKKPNAFIFISQRPSPETQ

SEQ ID NO: 15 - protein, 214 aa, Artificial
  HIL10R1
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 16 - protein, 434 aa, Artificial
  RhIL10R1
AHGTELPSPPSVWFEAEFFHHILHWTPIPNQSESTCYEVALLRYGTGRWNSISNCSQALSYDLTAVTLDLYRSNGYWARVRAVDGSRHSNWTVTNTRFSLDEVTLTVGSVKLEIHNGFILGKIQPPRPKMAPANDTYESIFRHFREYEIAIRKVPGNFTFTHKKVKHENFSLLTSGEVGEFCVQVKPSVTSRTNKGMWSKEECVSLTRQYFTVTNVGSGGGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLGGCGTPGK

SEQ ID NO: 17 - protein, 437 aa, Artificial
  RhIL10R1-SLS
AHGTELPSPPSVWFEAEFFHHILHWTPIPNQSESTCYEVALLRYGTGRWNSISNCSQALSYDLTAVTLDLYRSNGYWARVRAVDGSRHSNWTVTNTRFSLDEVTLTVGSVKLEIHNGFILGKIQPPRPKMAPANDTYESIFRHFREYEIAIRKVPGNFTFTHKKVKHENFSLLTSGEVGEFCVQVKPSVTSRTNKGMWSKEECVSLTRQYFTVTNVGSGGGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSGGCGTPGK

SEQ ID NO: 18 - protein, 57 aa, Artificial
  RPXV-UTR_170
TCAIRSKEDVPNFKDKMARVIKRKFNKQSQSYLTKFLGSTSNDVTTFLSMLNLTKYS

SEQ ID NO: 19 - protein, 57 aa, Artificial; identical to SEQ ID NO: 18
  HSPV-MNR76-211

SEQ ID NO: 20 - protein, 57 aa, Artificial
  VACV-Lister-257
TCAIRSKEDVPNFKDKMARVIKRKFNTQSQSYLTKFLGSTSNDVTTFLSMLNLTKYS

SEQ ID NO: 21 - protein, 52 aa, Artificial
  MPXV-ZRE_170
TCAIRSKEDVPNFKDKIARVITRKFNKQSQSYLTKFLGSTSNDVTTFLSILD

SEQ ID NO: 22 - protein, 57 aa, Artificial; identical to SEQ ID NO: 18
  VACV-COP_236

SEQ ID NO: 23 - protein, 57 aa, Artificial
  CPXV-GRI_188
TCAIRSKEDVPNFKDKMARVIKRKFNKQSQSYLTKFLGSTSNDVTTFLSMLDLTKYS

SEQ ID NO: 24 - protein, 52 aa, Artificial
  EVGBP
TCAIRSKEDVPNFKEKMTRVIKRKFNKQSHSYLTKFLGSTSNDITTFLSMLD

SEQ ID NO: 25 - protein, 52 aa, Artificial
  CPXV-BR_206
TCAIRSKEDVPNFKDKMARVIKRKFNKQSQSYLTKFLDSTSNDVMTVLSMLD

SEQ ID NO: 26 - protein, 52 aa, Artificial
  CPXV-GER91-189
TCAIRSKEDVSNFNDKMTRVIKRKFNKQSQNYMTKFLGTTANDVTTVLSMLD

SEQ ID NO: 27 - protein, 52 aa, Artificial
  CMLV-CMS_232
TCAIRSKEDVSNFKDKMTRVIKRKFNKQSQNYMTKFLGTTANDVTTVLSMLD

SEQ ID NO: 28 - protein, 52 aa, Artificial; identical to SEQ ID NO: 27
  TATV-DAH68-191

SEQ ID NO: 29 - protein, 52 aa, Artificial
  VARV-GAR_184
TCAIRSKEDVSNFKDKMTRVIKRKFNKQSQNYMTKFLGTTANDVTTVISMLD

SEQ ID NO: 30 - protein, 52 aa, Artificial; identical to SEQ ID NO: 29
  VARV-BOT72-181

SEQ ID NO: 31 - protein, 52 aa, Artificial; identical to SEQ ID NO: 29
  VARV-IND_177

SEQ ID NO: 32 - protein, 52 aa, Artificial; identical to SEQ ID NO: 29
  VARV-KUW67-181

SEQ ID NO: 33 - protein, 52 aa, Artificial; identical to SEQ ID NO: 29
  VARV-BSH_172

SEQ ID NO: 34 - protein, 52 aa, Artificial
  MYXV-LAU_161
PYIVKKSSDLEYVKRAIHNEYRLDTSSEGRRLEELYLTVASMFERLVEDVFE

SEQ ID NO: 35 - protein, 51 aa, Artificial
  SFV-KAS_157
PYIVKKPSDLEDVKRIISNEYRFDKTEERSRLEDLYLMIASMFQRLVEDIF

SEQ ID NO: 36 - protein, 57 aa, Artificial
  SWPV-NEB_008
TYFIKDFDDIDRVNNRLYRVVSDKYESNISSKFMHLYNNILSSFKLILQELMVNTEQ

SEQ ID NO: 37 - protein, 53 aa, Artificial
  DPV-W848_83-010
ICVLKSRNDIPYLKERVKNVVDKKYKKKCGPKLKVYELILSLLDEMLLDIGEN

SEQ ID NO: 38 - protein, 53 aa, Artificial
  DPV-W1170_84-010
MCVLKSRNDIPYLKERVKNIVDKKYKKKCGPKLKIYELILSLLDEMLLDIGEN

SEQ ID NO: 39 - protein, 53 aa, Artificial
  GTPV-Pellor_006
TFFVKQKTDLNLLKRRIRKVTKNKFKGEKSFNKIVGIYNLVLSLFEYIVNELN

SEQ ID NO: 40 - protein, 53 aa, Artificial
  LSDV-WARM_008
TFLVKQKTDLSLLKRRIQKVTKNKFKGEKSFNKIVGIYNLVLSLFEYIVNELN

SEQ ID NO: 41 - protein, 53 aa, Artificial
  SPPV-TU_006
TFFVKQKTDLSLLKRRIRKVTKNKFKGEKSFNKIVGIYNLVLSLFEYIVNELN

SEQ ID NO: 42 - protein, 53 aa, Artificial; identical to SEQ ID NO: 40
  LSDV-1959_008

SEQ ID NO: 43 - protein, 215 aa, Artificial
  Rhesus IgG1-FCh
PSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSGGCGTPGK

SEQ ID NO: 44 - DNA, 45 bp, Artificial
  primer
agtctgagtggaggttgcggaacaccgggtaaatgatgaaccggt

SEQ ID NO: 45 - DNA, 36 bp, Artificial
  primer
actcagactgaggctcttctgggtgtagtggttgtg

(19) United States
(12) Patent Application Publication
(10) Pub. No.: US 2022/0144911 A1
(43) Pub. Date: May 12, 2022

(54) METHODS AND COMPOSITIONS FOR IL10 SIGNALING ANTAGONISM

(71) Applicant: UAB Research Foundation, Birmingham, AL (US)

(72) Inventors: Mark R. Walter, Birmingham, AL (US); Ashlesha Deshpande, Vestavia Hills, AL (US)

(21) Appl. No.: 17/427,475

(22) PCT Filed: Jan. 30, 2020
(86) PCT No.: PCT/US2020/015845

§ 371 Date: Jul. 30, 2021

Related U.S. Application Data
(60) Provisional application No. 62/800,045, filed on Feb. 1, 2019.

(51) Int. Cl. C07K 14/54; A61K 47/65

(52) U.S. Cl. CPC C07K 14/5428; C07K 2319/30; C07K 2319/02; A61K 47/65

(57) ABSTRACT

This invention relates to fusion proteins, nucleic acid molecules, DNA constructs, and pharmaceutical compositions comprising the same, and methods of their use in IL10 antagonism.

### STATEMENT OF PRIORITY

This application claims the benefit, under 35 U.S.C. § 119(e), of U.S. Provisional Application No. 62/800,045, filed on Feb. 1, 2019, the entire contents of which are incorporated by reference herein.

### STATEMENT OF GOVERNMENT SUPPORT

This invention was made with government support under grant numbers R21AI081065 and R01AI049342 awarded by the National Institutes of Health. The government has certain rights in the invention.

### STATEMENT REGARDING ELECTRONIC FILING OF A SEQUENCE LISTING

A Sequence Listing in ASCII text format, submitted under 37 C.F.R. § 1.821, entitled 5656-70WO_ST25.txt, 59,485 bytes in size, generated on Jan. 28, 2020 and filed via EFS-Web, is provided in lieu of a paper copy. This Sequence Listing is hereby incorporated herein by reference into the specification for its disclosures.

### FIELD OF THE INVENTION

The present invention provides methods and compositions for interleukin 10 (IL10) signaling antagonism.

### BACKGROUND OF THE INVENTION

Vaccination with proteins or viruses can induce an immune response shaped by immunosuppressive factors rather than the desired immunostimulatory environment. As a result, the intended immune response to antigens within the vaccine does not occur. A major cytokine that contributes to the immunosuppressive phenotype is interleukin-10 (IL10). IL10 can be produced by cells of the host (cellular IL10) or by certain viruses, such as cytomegalovirus (CMV) and Epstein Barr Virus (EBV), which encode their own viral IL10. Both sources of IL10 are detrimental to immune responses, especially if the proteins are expressed before or in the initial stages of the immune response.

Modified CMV viruses have been shown to be very important in the development of vaccination strategies for simian immunodeficiency virus (SIV, HIV in humans) as well as tuberculosis. Thus, optimization of vaccine strategies could have a huge impact on many critical diseases that impact human health. However, difficulty developing effective broad-spectrum antagonism of IL10 signaling remains, and prior attempts to do so have been underpowered. There remains a need in the art for an effective IL10 signaling antagonist that can target the multiple potential sources of IL10. The present invention overcomes shortcomings in the art by providing fusion proteins, nucleic acid molecules, DNA constructs, and pharmaceutical compositions that function in IL10 antagonism.

### SUMMARY OF THE INVENTION

The present invention is based, in part, on the development of fusion proteins, nucleic acid molecules, DNA constructs, and pharmaceutical compositions for the purpose of providing an IL10 antagonist to a subject in need thereof. Thus, provided herein are fusion proteins that bind IL10 and inhibit IL10 signaling, comprising an IL10R1 domain, a linker peptide, and a multimerization domain, and methods of using the same.

One aspect of the invention provides a fusion protein comprising: a) an IL10R1 domain; b) a linker peptide; and c) a multimerization domain. In some embodiments, the fusion protein may further comprise a heterologous signal peptide. In some embodiments, the fusion protein may further comprise a dimer peptide.

A second aspect of the invention provides a dimer fusion protein comprising two fusion proteins of the present invention, wherein the two fusion proteins are′ covalently linked via the multimerization domains.

A third aspect of the invention provides a tetramer fusion protein comprising four fusion proteins of the present invention, wherein the four fusion proteins are covalently linked via the multimerization domains.

An additional aspect of the invention provides an octamer fusion protein comprising four fusion proteins and four different fusion proteins, wherein the fusion proteins are covalently linked to form an octamer fusion protein.

Another aspect of the invention relates to a nucleic acid molecule comprising a nucleotide sequence encoding a fusion protein of this invention, and DNA constructs and pharmaceutical compositions comprising the nucleic acid molecules and/or fusion proteins of the invention.

A further aspect of the invention provides a method of delivering a fusion protein to a subject, comprising administering to the subject a fusion protein, a dimer fusion protein, a tetramer fusion protein, an octamer fusion protein, a nucleic acid molecule, a DNA construct, and/or a pharmaceutical composition of the invention, thereby delivering the fusion protein to the subject.

Another aspect of the invention provides a method of inhibiting IL10 from inducing IL10 signaling in a cell, comprising contacting a fusion protein, a dimer fusion protein, a tetramer fusion protein, an octamer fusion protein, a nucleic acid molecule, a DNA construct, and/or a pharmaceutical composition of the invention, with a substrate comprising the cell and IL10, thereby inhibiting IL10 from inducing IL10 signaling in the cell.

A further aspect of the invention provides a method of inducing an immune response in a subject, comprising administering to the subject a fusion protein, a dimer fusion protein, a tetramer fusion protein, an octamer fusion protein, a nucleic acid molecule, a DNA construct, and/or a pharmaceutical composition of the invention, and an immunogen.

Another aspect of the invention provides a method of priming an immune response to an immunogen that induces an endogenous IL10 response in a subject, comprising: a) administering a fusion protein, a dimer fusion protein, a tetramer fusion protein, an octamer fusion protein, a nucleic acid molecule, a DNA construct, and/or a pharmaceutical composition of the invention, to the subject in an amount effective in inhibiting an endogenous IL10 response in the subject; and b) administering the immunogen.

A further aspect of the invention provides a method of enhancing an immune response to an immunogen that induces an IL10 response in a subject, comprising administering an effective amount of a fusion protein, a dimer fusion protein, a tetramer fusion protein, an octamer fusion protein, a nucleic acid molecule, a DNA construct, and/or a pharmaceutical composition of the invention, and the immunogen to a subject, wherein the immune response is enhanced in the subject as compared to an immune response in a subject to whom the immunogen is administered in the absence of the fusion protein, dimer fusion protein, tetramer fusion protein, octamer fusion protein, nucleic acid molecule, DNA construct, and/or pharmaceutical composition of the invention.

These and other aspects of the invention are set forth in more detail in the description of the invention below.

### BRIEF DESCRIPTION OF THE DRAWINGS

FIG. 1 shows the amino acid sequence of the basic RhIL10R1-FC (SEQ ID NO:12) prior to addition of the amino acid residues SLS.

FIG. 2 shows an amino acid sequence alignment of basic RhIL10R1 expressed in the RhIL10R1-FC (Rhehh10R1) (SEQ ID NO:13), RhIL10R1 WT sequence (Rhxp10R1) (SEQ ID NO:14), and the sequence of WT human IL10R1 (HIL10R1) (SEQ ID NO:15). Residue differences are bolded.

FIG. 3 shows the amino acid sequence of RhIL10R1 (SEQ ID NO:16) and of RhIL10R1-SLS (SEQ ID NO:17) with the three residue SLS insertion. RhIL10R1-SLS includes the RhIL10R1 and FC sequences.

FIG. 4 shows monomeric RhIL10R1, prior to insertion of the “SLS” sequence. Elutes were pooled (0.6 ml) and dialyzed against 4 L 20 mM Tris HCL pH 8.0 150 mM NaCl. The volume after dialysis was 1 ml. The molecular weight (MW) of the monomer was 56.00 KDa with 7 glycosylations, and the MW of the dimer was 112.00 KDa. Lane 1 is the marker (5 ul loaded); lane 2 is the wash (10 ul loaded, total volume 3 ml); and lane 3 is elute 1; lane 4 is elute 2; lane 4 is elute 3, lane 6 is elute 4, lane 7 is blank; lane 8 is elute 2 and BME. Lanes 3-8 were each loaded with 10 ul protein for a total volume of 0.2 ml.

FIG. 5 shows initial purification of RhIL10R1-FC with addition of SLS. Hereinafter RhIL10R1-FC is referred to interchangeably as RhIL10R1-FC-SLS and/or RhIL10R1-FC. SDS-PAGE gel of RhIL10R1-FC characterized in the absence (oxidized) or presence (reduced) of the reducing agent dithiothreitol (DTT). This gel confirms the covalent linkage of two RhIL10R1-FC molecules to make a dimeric RhIL10R1-FC with an approximate molecular weight of 102,000 KDa.

FIG. 6 shows analysis of RhIL10R1-FC and 1F11 by surface plasmon resonance (SPR). RhIL10R1-FC or the anti-IL10 antibody (Ab) 1F11 was captured to a biacore chip using an anti-human FC capture kit from GE Life Sciences. IL10s were injected over RhIL10R1-FC and 1F11 at a flow rate of 100 μL/min for 120 seconds. Following the association phase, dissociation was monitored for 720 seconds. The binding parameters were extracted from the sensorgrams by fitting the data to a 1:1 binding model. Binding constants derived from the sensorgrams are shown in Table 3.

FIG. 7 shows restoration of RhIL12 production by LPS-stimulated monocytes using RhIL10R1-FC (FIG. 7, left panel) or Ab 1F11 (FIG. 7, right panel). RhIL12 levels (y-axis) from the cell supernatants were measured by ELISA from six animals, following treatment with 10 ng/mL RhIL10 only, or with increasing concentrations of RhIL10R1-FC or Ab 1F11 (x-axis). The ELISA data are normalized relative to the amount of RhIL12 produced in the absence of IL10 (e.g., media only, or y-axis values=RhIL12^{(RhIL10+RhIL10R1-FC)}/RhIL12^{(media only)}). Y-axis values greater than or equal to 1 infer complete neutralization of IL10 activity.

FIG. 8 shows restoration of RhIL12 production by LPS-stimulated monocytes using RhIL10R1-FC (FIG. 8, left panel) or Ab 1F11 (FIG. 8, right panel). RhIL12 levels (y-axis) from the cell supernatants were measured by ELISA from six animals, following treatment with 10 ng/mL RhCMVIL10 only, or with increasing concentrations of RhIL10R1-FC or Ab 1F11 (x-axis). The ELISA data are normalized relative to the amount of RhIL12 produced in the absence of IL10 (e.g., media only, or y-axis values=RhIL12^{(RhIL10+RhIL10R1-FC)}/RhIL12^{(media only)}) Y-axis values greater than or equal to 1 infer complete neutralization of IL10 activity.

FIG. 9 shows restoration of RhIL12 production by LPS-stimulated monocytes using RhIL10R1-FC. RhIL12 levels (y-axis) from the cell supernatants were measured by ELISA from six animals, following treatment with 10 ng/mL human IL10 only, or with increasing concentrations of RhIL10R1-FC or Ab 1F11 (x-axis). The ELISA data are normalized relative to the amount of RhIL12 produced in the absence of IL10 (e.g., media only, or y-axis values=RhIL12^{(RhIL10+RhIL10R1-FC)}/RhIL12^{(media only)}). Y-axis values greater than or equal to 1 infer complete neutralization of IL10 activity.

FIG. 10 shows the oligomeric structure of the IL10/IL10R1 complex as observed in the crystal structure of the complex. IL10R1s are the β-strand containing proteins indicated with arrows.

FIG. 11 shows the tetrameric structure of the EVGBP. The D3 oligomerization domain is circled.

FIG. 12 shows an alignment of D3 domain sequences of multiple IFNγBP proteins. The location of residues that have been mutated is numbered. In addition to EVGBP, other D3 peptides with unique sequence features are underlined. Sequences shown include RPXV-UTR_170 (SEQ ID NO:18), HSPV-MNR76-211 (SEQ ID NO:19), VACV-Lister-257 (SEQ ID NO:20), MPXV-ZRE_170 (SEQ ID NO:21), VACV-COP_236 (SEQ ID NO:22), CPXV-GRI_188 (SEQ ID NO:23), EVGBP (SEQ ID NO:24), CPXV-BR_206 (SEQ ID NO:25), CPXV-GER91-189 (SEQ ID NO:26), CMLV-CMS_232 (SEQ ID NO:27), TATV-DAH68-191 (SEQ ID NO:28), VARV-GAR 184 (SEQ ID NO:29), VARV-BOT72-181 (SEQ ID NO:30), VARV-IND_177 (SEQ ID NO:31), VARV-KUW67-181 (SEQ ID NO:32), VARV-BSH_172 (SEQ ID NO:33), MYXV-LAU_161 (SEQ ID NO:34), SFV-KAS_157 (SEQ ID NO:35), SWPV-NEB_008 (SEQ ID NO:36), DPV-W848_83-010 (SEQ ID NO:37), DPV-W1170_84-010 (SEQ ID NO:38), GTPV-Pellor_006 (SEQ ID NO:39), LSDV-WARM_008 (SEQ ID NO:40), SPPV-TU_006 (SEQ ID NO:41), and LSDV-1959_008 (SEQ ID NO:42).

FIG. 13 shows an analysis of MBP-D3 fusion protein assembly in the presence or absence of reducing agent DTT. The results show Cys216 is not required for MDP-D3 tetramer formation.

FIG. 14 shows assembly properties of EVGBP D3 mutants and deer poxvirus (DPV) D3 peptide.

FIG. 15 shows a schematic diagram of methods for assembling IL10R1 multimers of 4 and 8 using the EVGBP-D3 domain.

FIG. 16 shows a schematic diagram of methods of assembling dimer, tetramer, and octamer fusion proteins using the FC and/or EVGBP-D3 domains.

FIG. 17 shows the sequence of Rhesus IgG1-FCh (SEQ ID NO:43). Residues 1-207 are of RhIgG1 (e.g., SEQ ID NO:10), and residues 208-215 are an added dimer sequence. Residues marked with an asterisk indicate locations of modifications to make hole and/or knob configurations. In some embodiments, an R118C substitution and a T129Y substitution may be made to modify the FC for “knob” (“FCk”). In some embodiments, a Y170T substitution may be made to modify the FC for “hole” (FCh). An example addition of a dimer peptide (e.g., c-terminal addition of GGCGTPGK (SEQ ID NO:3)) made to the FCh is shown.

### DETAILED DESCRIPTION OF THE INVENTION

The present invention is based on the unexpected discovery that multimeric IL10R1 fusion proteins can be generated to neutralize human, rhesus macaque, CMV and other sourced IL10 molecules, to inhibit endogenous IL10 signaling.

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for production of recombinant and synthetic genes, polypeptides, proteins, manipulation of nucleic acid sequences, production of transformed cells, and the construction of vector constructs. Such techniques are known to those skilled in the art. See, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

### Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

“Optional” or “optionally” means that the subsequently described event or circumstance can or cannot occur, and that the description includes instances where the event or circumstance occurs and instances where it does not.

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or less additional nucleotides or amino acids includes the total number of additional nucleotides or amino acids added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in biological activity of at least about 50% or more as compared to the activity of a polypeptide consisting of the recited sequence.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol. 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

Furthermore, an “isolated” cell is a cell that has been partially or completely separated from other components with which it is normally associated in nature. For example, an isolated cell can be a cell in culture medium and/or a cell in a pharmaceutically acceptable carrier.

The term “endogenous” refers to a component naturally found in an environment, i.e., a gene, nucleic acid, miRNA, protein, cell, or other natural component expressed in the subject, as distinguished from an introduced component, i.e., an “exogenous” component.

As used herein, the term “heterologous” refers to a nucleotide/polypeptide that originates from a foreign species, or, if from the same species, is substantially modified from its native form in composition and/or genomic locus by deliberate human intervention.

As used herein, the term “nucleic acid” refers to a single or double-stranded polymer of deoxyribonucleotide or ribonucleotide bases read from the 5′ to the 3′ end. The “nucleic acid” may also optionally contain non-naturally occurring or modified nucleotide bases. The term “nucleotide sequence” or “nucleic acid sequence” refers to both the sense and antisense strands of a nucleic acid as either individual single strands or in the duplex.

The terms “nucleic acid segment,” “nucleotide sequence,” “nucleic acid molecule,” or more generally “segment” will be understood by those in the art as a functional term that includes both genomic DNA sequences, ribosomal RNA sequences, transfer RNA sequences, messenger RNA sequences, small regulatory RNAs, operon sequences and smaller engineered nucleotide sequences that express or may be adapted to express, proteins, polypeptides or peptides. Nucleic acids of the present disclosure may also be synthesized, either completely or in part, by methods known in the art. Thus, all or a portion of the nucleic acids of the present codons may be synthesized using codons preferred by a selected host. Species-preferred codons may be determined, for example, from the codons used most frequently in the proteins expressed in a particular host species. Other modifications of the nucleotide sequences may result in mutants having slightly altered activity.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” or “active fragment” refers to nucleic acid that encodes a functional fragment of a polypeptide.

As used herein with respect to polypeptides, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., the ability to up- or down-regulate gene expression). In some embodiments, the functional fragment actually has a higher level of at least one biological activity of the full-length polypeptide.

As used herein, the term “modified,” as applied to a polynucleotide or polypeptide sequence, refers to a sequence that differs from a wild-type sequence due to one or more deletions, additions, substitutions, or any combination thereof. Modified sequences may also be referred to as “modified variant(s).”

As used herein, by “isolate” or “purify” (or grammatical equivalents) a vector, it is meant that the vector is at least partially separated from at least some of the other components in the starting material.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts may be referred to as “transcription products” and encoded polypeptides may be referred to as “translation products.” Transcripts and encoded polypeptides may be collectively referred to as “gene products.” If the polynucleotide is derived from genomic DNA, expression may include splicing of the mRNA in a eukaryotic cell. The expression product itself, e.g., the resulting nucleic acid or protein, may also be said to be “expressed.” An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

As used herein, the term “fusion protein” refers to an amino acid sequence (e.g., polypeptide) generated non-naturally by deliberate human design comprising, among other components, an amino acid sequence of a protein of interest and/or a modified variant and/or active fragment thereof, linked to other amino acid segments. The different components of the protein may provide differing and/or combinatorial function. Structural and functional components of the fusion protein may be incorporated from differing and/or a plurality of source material. The fusion protein may be delivered exogenously to a subject, wherein it would be exogenous in comparison to a corresponding endogenous protein.

The terms “amino acid sequence,” “polypeptide,” “peptide” and “protein” may be used interchangeably to refer to polymers of amino acids of any length. The terms “nucleic acid,” “nucleic acid sequence,” and “polynucleotide” may be used interchangeably to refer to polymers of nucleotides of any length. As used herein, the terms “nucleotide sequence,” “polynucleotide,” “nucleic acid sequence,” “nucleic acid molecule” and “nucleic acid fragment” refer to a polymer of RNA, DNA, or RNA and DNA that is single- or double-stranded, optionally containing synthetic, non-natural and/or altered nucleotide bases.

As used herein, the terms “gene of interest,” “nucleic acid of interest” and/or “protein of interest” refer to that gene/nucleic acid/protein desired under specific contextual conditions.

As used herein with respect to nucleic acids, the term “operably linked” refers to a functional linkage between two or more nucleic acids. For example, a promoter sequence may be described as being “operably linked” to a heterologous nucleic acid sequence because the promoter sequences initiates and/or mediates transcription of the heterologous nucleic acid sequence. In some embodiments, the operably linked nucleic acid sequences are contiguous and/or are in the same reading frame.

A peptide of the invention may be sourced from heterologous peptides known or putatively validated in nature and directed toward a specific function and/or synthetically generated. As used herein, the term “signal peptide” refers to an amino acid sequence known in nature and/or generated synthetically with functionality for regulating protein expression levels and protein localization. For example, a signal peptide from a heterologous protein may be incorporated into and operably linked to a fusion protein comprising a protein, modified variant and/or active fragment of interest, in order to increase protein expression of the protein, modified variant and/or active fragment relative to its expression when operably linked to its wildtype signal peptide. A signal peptide may be referred to interchangeably as a signal sequence, targeting signal, localization sequence, and/or leader sequence/peptide.

As used herein, the term “dimer peptide” refers to an amino acid sequence with functionality to allow dimerization between two components, for example, between two dimer peptides. In some embodiments, a dimer peptide may comprise an amino acid sequence which provides specific amino acids such that a covalent bond can form between it and another amino acid sequence (e.g., a second dimer peptide, and/or a second component with a complementary dimer peptide binding site).

As used herein, the term “linker peptide” refers an amino acid sequence used to link two or more components together. Linker peptides may also provide additional functionality, such as flexibility and/or cleavability by enzymes present endogenously or added exogenously to an environment. Linker peptides may be generated synthetically, designed empirically and/or derived from naturally-occurring multiple-domain proteins.

As used herein, the term “domain” refers to a specified section of an amino acid sequence molecule (e.g., a protein) that forms a tertiary structure and/or provides a specialized function independent of the longer amino acid sequence within which it is a part. Domains may be independently stable and/or folded, and may be conserved in either consensus sequence and/or consensus tertiary structure with similar domains in other related and unrelated polypeptides or proteins.

As used herein, the term “multimerization domain” refers to a protein domain capable of multimerizing between itself, i.e., forming larger structures through interactions with multiple copies of the multimerization domain. The ability to form specific numbers of multimers (e.g., dimers, trimers, tetramers, hexamers, septamers, octamers, nonamers, etc.) may depend on the specific domain. For example, a tetramerization domain may be capable of forming tetramers.

As used herein, the term “FC domain” refers to the structural component of an immunoglobulin (Ig) molecule (i.e., an antibody) that is more readily crystallizable due to its stability (thus labeled “fragment crystallizable (“FC”)). A typical Ig molecule structure is comprised of two portions (e.g., domains), the FC portion and the Fab (“Fragment antigen-binding”) portion, wherein each molecule typically comprises 1 FC portion to every 2 Fab portions. In general, antibody specificity and antigen binding capacity is encoded within the Fab portion, which can be highly variable, while receptor specificity and receptor binding capacity is encoded within the FC portion, which is less variable and more conserved across related species. Relation in Ig FC portions generates classifications of Ig molecule, for example, wherein all IgG molecules have similar FC portions that can bind to IgG receptor molecules.

The term “covalent bond” or “covalent link(age)” refers to a chemical bond wherein atoms share electrons leading to a stable balance of attractive and repulsive forces and stable association between the sharing atoms. Two components associated together via a covalent bond can be referred to as “covalently coupled, “covalently linked” and/or “covalently bonded.” One example of a covalent bond is a disulfide bond, a covalent bond typically between two thiol groups each comprising a sulfur atom bound to a hydrogen and a carbon, whereupon binding, a covalent bond forms between the two sulfur atoms in the oxidation of the two hydrogen atoms. Thiol groups are commonly found on certain amino acids, e.g. cysteines. The directional planes of the atoms in respect to each other within an amino acid sequence are described as phi, psi, and omega dihedral angles.

The terms “mutation,” “mutant” and other grammatical variants encompass, at the amino acid sequence level of a fusion protein of this invention, any substitution with any naturally occurring amino acid residue (Table 1), any substitution with any non-naturally occurring amino acid residue (e.g., as listed in Table 2), any deletion, any insertion, and any combination thereof in a wild type amino acid sequence of a protein of interest (e.g., an IL10R1 protein, e.g., an IgG protein, e.g., a virus IFNγBP protein). These terms are also intended to encompass the incorporation of additional glycosylation sites into the fusion protein of this invention, as well as modifications in the amino acid sequence of the fusion protein that result in an alteration of the framework of the a protein of interest. These mutations can be introduced at the nucleic acid level by altering or modifying the nucleotide sequence encoding the fusion protein (e.g., to introduce into the nucleotide sequence a deletion, substitution, insertion, stop codon, missense mutation, nonsense mutation, etc.) according to well-known methods to produce the desired mutation at the amino acid sequence level.

Modifications/mutations that are substitutions may be commonly annotated as to their initial amino acid relative to the reference, and the modified amino acid, (e.g., F250A being an alanine replacing a phenylalanine at position 250 of an amino acid sequence, relative to a reference sequence). Modifications may or may not alter the functionality of the sequence and/or sequence product. For example a modification may prevent multimerization of a sequence or sequence product. In some embodiments, a modification that is a substitution may prevent a tetramerization domain from tetramerizing, and instead only form monomers and/or dimers.

By the term “treat,” “treating,” or “treatment of (or grammatically equivalent terms) it is meant that the severity of the subject's condition is reduced or at least partially improved or ameliorated and/or that some alleviation, mitigation or decrease in at least one clinical symptom is achieved and/or there is a delay in the progression of the condition and/or prevention or delay of the onset of a disease or disorder.

As used herein, the term “prevent,” “prevents,” or “prevention” (and grammatical equivalents thereof) refers to a delay in the onset of a disease or disorder or the lessening of symptoms upon onset of the disease or disorder. The terms are not meant to imply complete abolition of disease and encompasses any type of prophylactic treatment that reduces the incidence of the condition or delays the onset and/or progression of the condition.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The term “administering” or “administration” of a fusion protein, dimer fusion protein, tetramer fusion protein, octamer fusion protein, expression cassette, vector, DNA construct, plasmid, viral vector, transformed cell, nanoparticle, or pharmaceutical composition to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

As used herein, the term “antigen” refers to a molecule capable of inducing the production of immunoglobulins (e.g., antibodies). The term “immunogen” can be used interchangeably with “antigen” under certain conditions, e.g., when the antigen is capable of inducing a multi-faceted humoral and/or cellular-mediated immune response. A molecule capable of antibody and/or immune response stimulation may be referred to as antigenic/immunogenic, and can be said to have the ability of antigenicity/immunogenicity. The binding site for an antibody within an antigen and/or immunogen may be referred to as an epitope (e.g., an antigenic epitope).

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

A “subject” of the invention may include any animal in need thereof. In some embodiments, a subject may be, for example, a mammal, a reptile, a bird, an amphibian, or a fish. A mammalian subject may include, but is not limited to, a laboratory animal (e.g., a rat, mouse, guinea pig, rabbit, primate, etc.), a farm or commercial animal (e.g., cattle, pig, horse, goat, donkey, sheep, etc.), or a domestic animal (e.g., cat, dog, ferret, gerbil, hamster etc.). In some embodiments, a mammalian subject may be a primate, or a non-human primate (e.g., a chimpanzee, baboon, macaque (e.g., rhesus macaque, crab-eating macaque, stump-tailed macaque, pig-tailed macaque), monkey (e.g., squirrel monkey, owl monkey, etc.), marmoset, gorilla, etc.). In some embodiments, a mammalian subject may be a human. In some embodiments, a bird may include, but is not limited to, a chicken, a duck, a turkey, a goose, a quail, a pheasant, a parakeet, a parrot, a macaw, a cockatoo, or a canary.

A “subject in need” of the methods of the invention can be any subject known to have an illness to which inhibition of IL10 signaling may provide beneficial health effects, or a subject having an increased risk of developing the same (e.g., a subject having, for example, an HIV infection, an EBV infection, a CMV infection, malaria, tuberculosis, cancer, or any combination thereof).

### Fusion Proteins

This invention relates to fusion proteins, nucleic acid molecules, DNA constructs, and pharmaceutical compositions for the purpose of providing an IL10 antagonist to a subject in need thereof.

Thus, one aspect of the invention provides a fusion protein comprising a) an IL10R1 domain; b) a linker peptide; and c) a multimerization domain.

In some embodiments, the fusion protein may further comprise a heterologous signal peptide. The heterologous signal peptide comprises any signal peptide from viral, prokaryotic, or eukaryotic origin that can be used to direct secretory expression of the fusion protein. Many signal peptides are known in the art, such as prokaryotic signal sequences such as, for example, pelB or ompA. Examples of eukaryotic signal sequences include the cognate signal sequence of the protein of interest, modified cognate signal sequences, or heterologous signal sequences. Additionally, signal peptides can be predicted and/or optimized via known methods, such as for example, the use of computational methods such as SignalP 4.1 software. In some embodiments, the signal peptide is a heterologous signal peptide of an interferon gamma receptor 2 molecule (IFNγR2). In some embodiments, the signal peptide of the present invention comprises, consists essentially of, or consists of the nucleotide sequence of SEQ ID NO:1 or a peptide at least 70% identical thereto, e.g., at least about 70, 75, 80, 85, 90, 91, 92, 93, 94, 95, 96, 97, 98, or 99% identical thereto. In some embodiments, the heterologous signal peptide comprises SEQ ID NO:1, a signal peptide from IFNγR2, or any combination thereof.

IFNγR2 heterologous signal peptide
SEQ ID NO: 1
MLPRLVVLLAAFLSRRLGSDA

In some embodiments, the linker peptide of the present invention comprises any synthetic linker peptide known in the art or later identified. In some embodiments, the linker peptide comprises, consists essentially of, or consists of the nucleotide sequence of SEQ ID NO:2 or a peptide at least about 70% identical thereto, e.g., at least about 70, 75, 80, 85, 90, 91, 92, 93, 94, 95, 96, 97, 98, or 99% identical thereto.

Linker peptide
SEQ ID NO: 2
GSGGGG

The multimerization domain may be any functional protein domain that allows for multimerization of the fusion protein, e.g., for dimerization, tetramerization, or octamerization (i.e., 8mer) of the fusion protein. In some embodiments, the multimerization domain of the present invention may comprise the FC domain of an IgM molecule (e.g., an IgM antibody). In some embodiments, the multimerization domain of the present invention can comprise the FC domain of an IgG molecule (e.g., an IgG antibody). Examples of IgG molecules include, but are not limited to, IgG1, IgG2, IgG3, IgG4, and modified variants and/or active fragments thereof. Examples of modified variants of FC domains include, but are not limited to, FC “knob” (FCk) and FC “hole” (Fch) modified FCs as described below. IgG molecules can be found in any mammal, including humans and non-human mammals. Examples of non-human mammals include but are not limited to rhesus macaques, crab-eating macaques, stump-tailed macaques, pig-tailed macaques, squirrel monkeys, owl monkeys, baboons, chimpanzees, marmosets, spider monkeys, mice, pigs, rabbits, sheep, hamsters, guinea pigs, rats, or ferrets. In some embodiments, the FC domain is a domain from a human or a rhesus macaque.

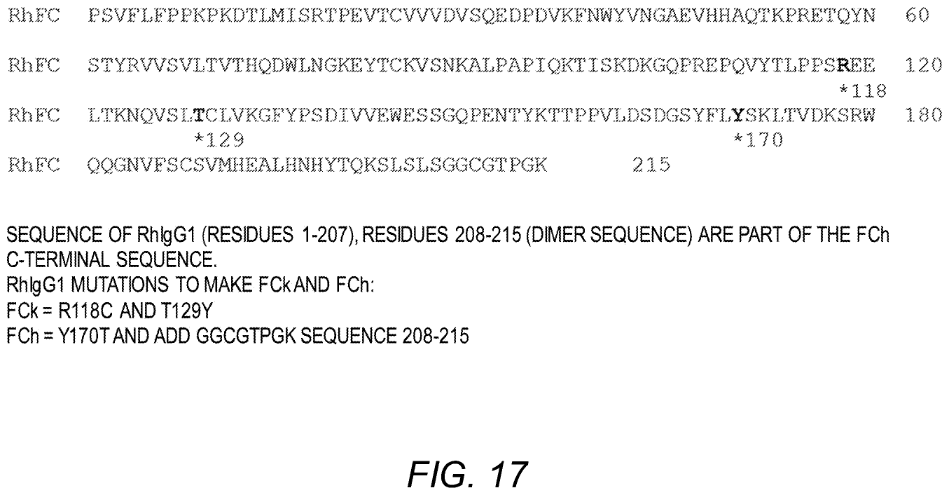

In some embodiments, the FC domain may be modified with substitution mutations to modify the function and/or binding capabilities of the FC domain. For example, the FC domain may be modified to allow binding with an apposing alternative FC domain and/or prevent binding with a matching FC domain (e.g., binding of a knob (“k”) A and hole (“h”) B as A+B (“hk”) and preventing A+A (“hh”) or B+B “kk”). Knob and hole modifications are further described in Ridgway et al. Protein Engineering 9(7):617-621 (1996); and Deshpande et al. Protein Science 22:1100-1108 (2013), the relevant disclosures of which are incorporated herein by reference. Non-limiting examples of an FC domain mutation of the present invention include Y170T, T129Y, and R118C mutations, wherein the numbering starts at the initial proline of the wildtype FC domain sequence (FIG. 17 and SEQ ID NO:10). Thus, in some embodiments, the present invention provides a fusion protein wherein the FC domain is a modified FC domain with a Y170T, T129Y, and/or R118C mutation.

Rhesus IgG1 FC domain corresponding to FIG. 17
(residues 1-207)
SEQ ID NO: 10
psvflfp pkpkdtlmis rtpevtcvvv dvsqedpdvk
fnwyvngaev hhaqtkpret qynstyrvvs vltvthqdwl
ngkeytckvs nkalpapiqk tiskdkgqpr epqvytlpps
reeltknqvs ltclvkgfyp sdivvewess gqpentyktt
ppvldsdgsy flyskltvdk srqqqgnvfs csvmhealhn
hytqksl

In some embodiments, the present invention comprises a multimerization domain further comprising a dimer peptide. A dimer peptide of the present invention can be any dimer that provides functionality for covalent bonding between itself and another component, e.g. a second dimer peptide or a second component with a dimer peptide binding site. In some embodiments, the dimer peptide comprises, among other amino acids, two glycine (G) residues that do not have constrained phi/psi angles before and after cysteine (C) residue for optimal disulfide covalent bonding. In some embodiments, the dimer peptide of the present invention comprises, consists essentially of, or consists of the nucleotide sequence of SEQ ID NO:3 or a peptide at least 70% identical thereto, e.g., at least about 70, 75, 80, 85, 90, 91, 92, 93, 94, 95, 96, 97, 98, or 99% identical thereto. In some embodiments, the dimer peptide of the present invention comprises, consists essentially of, or consists of the nucleotide sequence of SEQ ID NO:11 or a peptide at least 70% identical thereto, e.g., at least about 70, 75, 80, 85, 90, 91, 92, 93, 94, 95, 96, 97, 98, or 99% identical thereto.

Dimer Peptide
SEQ ID NO: 3
GGCGTPGK
Dimer Peptide
SEQ ID NO: 11
GGCGTPAS

In some embodiments of the present invention, the fusion protein comprises a heterologous IFNγR2 signal peptide, an IL10R1 domain of a rhesus macaque, a linker peptide, an FC domain of an IgG1 molecule of a rhesus macaque, and a dimer peptide. In some embodiments of the present invention, the fusion protein comprises a heterologous signal peptide, an IL10R1 domain, a linker peptide, an FC domain of an IgG molecule, and a dimer peptide, that comprises, consists essentially of, or consists of the nucleotide sequence SEQ ID NO:4 or a nucleotide at least 70% identical thereto, e.g., at least about 70, 75, 80, 85, 90, 91, 92, 93, 94, 95, 96, 97, 98, or 99% identical thereto.

RhesusIL10R1-RhesusFC
SEQ ID NO: 4
MLPRLVVLLAAFLSRRLGSDAhgtelpspp svwfeaeffh
hilhwtpipn qsestcyeva llrygtgrwn sisncsqals
ydltavtidl yrsngywarv ravdgsrhsn
wtvtntrfsldevtltvgsv kleihngfil gkiqpprpkm
apandtyesi frhfreyeia irkvpgnftfthkkvkhenf
slltsgevge fcvqvkpsvt srtnkgmwsk eecvsltrqy
ftvtnv GSGGGGpsvflfp pkpkdtlmis rtpevtcvvv
dvsqedpdvk fnwyvngaev hhaqtkpret qynstyrvvs
vltvthqdwl ngkeytckvs nkalpapiqk tiskdkgqpr
epqvytlpps reeltknqvs ltclvkgfyp sdivvewess
gqpentyktt ppvldsdgsy flyskltvdk srqqqgnvfs
csvmhealhn hytqkslGGCGTPGK*

In some embodiments of the present invention, the fusion protein comprises a heterologous IFNγR2 signal peptide, an IL10R1 domain of a human, a linker peptide, an FC domain of an IgG1 molecule of a human, and a dimer peptide. In some embodiments of the present invention, the fusion protein comprises a heterologous signal peptide, an IL10R1 domain, a linker peptide, an FC domain of an IgG molecule, and a dimer peptide, that comprises, consists essentially of, or consists of the nucleotide sequence SEQ ID NO:5 or a nucleotide at least 70% identical thereto, e.g., at least about 70, 75, 80, 85, 90, 91, 92, 93, 94, 95, 96, 97, 98, or 99% identical thereto.

IL10R1-HumanFC
SEQ ID NO: 5
MLPRLVVLLAAFLSRRLGSDA HGTELPSPP SVWFEAEFFH
HILHWTPIPN QSESTCYEVA LLRYGIESWN SISNCSQTLS
YDLTAVTLDL YHSNGYRARV RAVDGSRHSN WTVTNTRFSV
DEVTLTVGSV NLEIHNGFIL GKIQLPRPKM APANDTYESI
FSHFREYEIA IRKVPGNFTF THKKVKHENF SLLTSGEVGE
FCVQVKPSVA SRSNKGMWSK EECISLTRQY FTVTNV GSGGGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSRDE
LTKNQVSLTC LVKGFYPSDI AVEWESNGQP ENNYKTTPPV
LDSDGSFFLY SKLTVDKSRW QQGNVFSCSV MHEALHNHYT
QKSLSLS GGCGTPGK*

In some embodiments of the present invention, the fusion protein comprises a heterologous IFNγR2 signal peptide, an IL10R1 domain of a rhesus macaque, a linker peptide, an FC domain of an IgG1 molecule of a rhesus macaque that is modified with a “hole” mutation, and a dimer peptide. In some embodiments of the present invention, the fusion protein comprises a heterologous signal peptide, an IL10R1 domain, a linker peptide, an FC domain of an IgG molecule, and a dimer peptide, that comprises, consists essentially of, or consists of the nucleotide sequence SEQ ID NO:6 or a nucleotide at least 70% identical thereto, e.g., at least about 70, 75, 80, 85, 90, 91, 92, 93, 94, 95, 96, 97, 98, or 99% identical thereto.

RhIL10R1-RhFCh
SEQ ID NO: 6
MLPRLVVLLAAFLSRRLGSDA hgtelpspp svwfeaeffh
hilhwtpipn qsestcyeva llrygtgrwn sisncsqals
ydltavtldl yrsngywarv ravdgsrhsn
wtvtntrfsldevtltvgsv kleihngfil gkiqpprpkm
apandtyesi frhfreyeia irkvpgnftfthkkvkhenf
slltsgevge fcvqvkpsvt srtnkgmwsk eecvsltrqy
ftvtnv GSGGGG psvflfp pkpkdtlmis rtpevtcvvv
dvsqedpdvk fnwyvngaev hhaqtkpret qynstyrvvs
vltythqdwl ngkeytckvs nkalpapiqk tiskdkgqpr
epqvytlpps reeltknqvs ltclvkgfyp sdivvewess
gqpentyktt ppvldsdgsy flTskltvdk srqqqgnvfs
csvmhealhn hytqkslsls GGCGTPGK*

In some embodiments of the present invention, the multimerization domain may be an IFNγ binding protein, modified variants thereof or active fragments thereof. The IFNγ binding protein (IFNγBP) may be any known or putative IFNγ binding protein, such as, for example, an IFNγBP of an animal, plant, virus, or bacterium. In some embodiments, the IFNγBP may a modified variant and/or active fragment thereof. In some embodiments, the IFNγBP may be an IFNγ protein of a human or a non-human primate (e.g., a rhesus macaque). In some embodiments, the IFNγBP may be an IFNγBP of a virus, for example, an IFNγBP from a poxvirus. Non-limiting examples of poxviruses with IFNγBPs include ectromelia virus (EV), myxoma virus (MYXV), or deerpox virus (DPV). In some embodiments, the IFNγBP, modified variant thereof, or active fragment thereof, is EVGBP, D3-EVGBP, MYXV-LAU_171, DPV-W949_93-011, or any combination thereof.

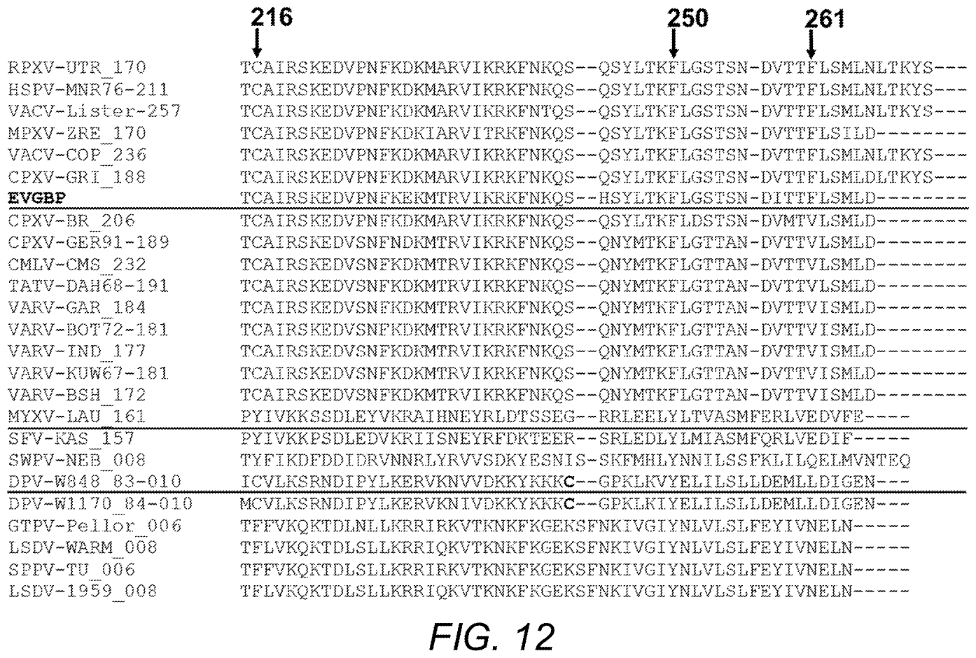

In some embodiments, the IFNγBP tetramerization domain may have a modification that alters the ability to multimerize, for example, to only allow monomer formation, and/or to only allow dimer formation. Non-limiting examples of D3 mutations in the IFNγBP tetramerization domain include F261P and F250A, wherein the numbering corresponds to the aligned D3 domains of wildtype full length IFNγBP sequences as shown in FIG. 12. In some embodiments, an F261P mutation prevents tetramerization and allows monomer formation by the modified D3 domain. In some embodiments, an F250A mutation prevents tetramerization and allows dimer formation by the modified D3 domain. In some embodiments, the modified IFNγBP tetramerization domain (e.g., the modified D3 domain) may be the D3 domain of EVGBP. Thus, in some embodiments, the present invention provides a fusion protein wherein the IFNγ binding protein, modified variant thereof, or active fragment thereof, is a modified D3-EVGBP with an F261P mutation. In some embodiments, the present invention provides a fusion protein wherein the IFNγ binding protein, modified variant thereof, or active fragment thereof, is a modified D3-EVGBP with an F250A mutation.

In some embodiments, the IL10R1 domain of the present invention may be an IL10R1 domain of a human or a non-human mammal. In some embodiments, the IL10R1 domain is a domain from a human or a rhesus macaque. In some embodiments, the IL10R1 domain may comprise a fusion protein of the present invention, wherein the incorporated fusion protein does not comprise a heterologous signal peptide. In some embodiments, the IL10R1 domain may comprise a fusion protein comprising an IL10R1 domain, a linker peptide, and a multimerization domain. In some embodiments, the IL10R1 domain may comprise a fusion protein comprising an IL10R1 domain, a linker peptide, and an FC domain. In some embodiments, the FC domain of the incorporated fusion protein may be a modified FC domain to allow binding with an apposing (e.g., knob “k” or hole “h”) FC domain, e.g., modified with a knob “k” modification (e.g., FCk). In some embodiments, the FC domain of the incorporated fusion protein may be modified with a T129Y and/or an R118C mutation. In some embodiments, the IL10R1 domain may comprise a fusion protein comprising an IL10R1 domain, a linker peptide, and a modified FC domain with a T129Y and an R118C mutation.

In some embodiments of the present invention, the fusion protein comprises a heterologous IFNγR2 signal peptide, an IL10R1 domain of a rhesus macaque, a linker peptide, and an EVGBP D3 tetramerization domain. In some embodiments of the present invention, the fusion protein comprises a heterologous signal peptide, an IL10R1 domain, a linker peptide, and an EVGBP D3 tetramerization domain, that comprises, consists essentially of, or consists of the nucleotide sequence SEQ ID NO:7 or a nucleotide at least 70% identical thereto, e.g., at least about 70, 75, 80, 85, 90, 91, 92, 93, 94, 95, 96, 97, 98, or 99% identical thereto.

Rhesus IL10R1-D3EVGBP
SEQ ID NO: 7
MLPRLVVLLAAFLSRRLGSDA hgtelpspp svwfeaeffh
hilhwtpipn qsestcyeva llrygtgrwn sisncsqals
ydltavtldl yrsngywarv ravdgsrhsn
wtvtntrfsldevtltvgsv kleihngfil gkiqpprpkm
apandtyesi frhfreyeia irkvpgnftfthkkvkhenf
slltsgevge fcvqvkpsvt srtnkgmwsk eecvsltrqy
ftvtnv GSGGGG ytcairskedvpnfkekmtrvikrkfnkqshsyl
tkflgstsndittflsmld

In some embodiments of the present invention, the fusion protein comprises a heterologous IFNγR2 signal peptide, an IL10R1 domain of a human, a linker peptide, and an EVGBP D3 tetramerization domain. In some embodiments of the present invention, the fusion protein comprises a heterologous signal peptide, an IL10R1 domain, a linker peptide, and an EVGBP D3 tetramerization domain, that comprises, consists essentially of, or consists of the nucleotide sequence SEQ ID NO:8 or a nucleotide at least 70% identical thereto, e.g., at least about 70, 75, 80, 85, 90, 91, 92, 93, 94, 95, 96, 97, 98, or 99% identical thereto.

Human IL10R1-D3-EVGBP
SEQ ID NO: 8
MLPRLVVLLAAFLSRRLGSDA HGTELPSPP SVWFEAEFFH
HILHWTPIPN QSESTCYEVA LLRYGIESWN SISNCSQTLS
YDLTAVTLDL YHSNGYRARV RAVDGSRHSN WTVTNTRFSV
DEVTLTVGSV NLEIHNGFIL GKIQLPRPKM APANDTYESI
FSHFREYEIA IRKVPGNFTF THKKVKHENF SLLTSGEVGE
FCVQVKPSVA SRSNKGMWSK EECISLTRQY FTVTNV GSGGGG
Ytcairskedvpnfkekmtrvikrkfnkqshsyltkflgstsndittf
lsmld

In some embodiments of the present invention, the fusion protein comprises a heterologous IFN′R2 signal peptide; an incorporated fusion protein in place of an IL10R1 domain comprising a fusion protein comprising an IL10R1 domain, a linker peptide, and a modified FC domain with a T129Y and an R118C mutation; a linker peptide, and an EVGBP D3 tetramerization domain. In some embodiments of the present invention, the fusion protein comprises a heterologous signal peptide, an incorporated fusion protein in place of an IL10R1 domain, a linker peptide, and an EVGBP D3 tetramerization domain, that comprises, consists essentially of, or consists of the nucleotide sequence SEQ ID NO:9 or a nucleotide at least 70% identical thereto, e.g., at least about 70, 75, 80, 85, 90, 91, 92, 93, 94, 95, 96, 97, 98, or 99% identical thereto.

RhIL10R1-RhFCk-D3EVGBP
SEQ ID NO: 9
MLPRLVVLLAAFLSRRLGSDA hgtelpspp svwfeaeffh
hilhwtpipn qsestcyeva llrygtgrwn sisncsqals
ydltavtldl yrsngywarv ravdgsrhsn
wtvtntrfsldevtltvgsv kleihngfil gkiqpprpkm
apandtyesi frhfreyeia irkvpgnftfthkkvkhenf
slltsgevge fcvqvkpsvt srtnkgmwsk eecvsltrqy
ftvtnv GSGGGG psvflfp pkpkdtlmis rtpevtcvvv
dvsqedpdvk fnwyvngaev hhaqtkpret qynstyrvvs
vltvthqdwl ngkeytckvs nkalpapiqk tiskdkgqpr
epqvytlpps Ceeltknqvs lYclvkgfyp sdivvewess
gqpentyktt ppvldsdgsy flyskltvdk srqqqgnvfs
csvmhealhn hytqkslsls GSGGGG
ytcairskedvpnfkekmtrvikrkfnkqshsyltkflgstsndittf
lsmld*

Fusion proteins of the present invention may be multimerized in vitro and/or in vivo. For example, fusion proteins of the present invention may be dimerized (e.g., a dimer), tetramerized (e.g., a tetramer), and/or octamerized (e.g., an octamer, an 8mer) by expressing multiples of one or more of the fusion proteins of the present invention. In some embodiments, a dimer fusion protein may be formed by the combining (e.g., co-expressing) of at least two monomer fusion proteins comprising a heterologous signal peptide, an IL10R1 domain, a linker peptide, and a multimerization domain comprising an FC domain further comprising a dimer peptide. In some embodiments, a dimer fusion protein may be formed by the combining (e.g., co-expressing) of at least two monomer fusion proteins comprising, for example, SEQ ID NO:4 or SEQ ID NO:5.

In some embodiments, a dimer fusion protein of the present invention comprises two of the same fusion proteins of the present invention wherein the two fusion proteins are covalently linked via two dimer peptides. In some embodiments, a dimer fusion protein of the present invention comprises two different fusion proteins of the present invention wherein the two fusion proteins are linked via complementary knob and hole interactions. In some embodiments, a dimer fusion protein of the present invention comprises two different fusion proteins of the present invention wherein the two fusion proteins are linked via a dimer peptide and a complementary dimer peptide binding site.

In some embodiments, a tetramer fusion protein of the present invention comprises four of the same fusion proteins of the present invention wherein the four fusion proteins are linked via the four IFNγBP domains.

In some embodiments, an octamer fusion protein of the present invention comprises two different fusion proteins of the present invention (e.g., four of one fusion protein, and four of another fusion protein) wherein the two different fusion proteins are linked via a dimer peptide and a complementary dimer peptide binding site, and/or wherein one of the four of one fusion protein are linked via the four IFNγBP domains. In some embodiments, an octamer fusion protein of the present invention comprises two different fusion proteins of the present invention (e.g., four of one fusion protein, and four of another fusion protein), wherein the four and four different fusion proteins are covalently linked via their different knob and hole modified FC domains, and wherein the four same fusion proteins comprising a IFNγBP binding protein, modified variant thereof or active fragment thereof, are covalently linked via the IFNγBP binding protein, modified variant thereof or active fragment thereof, to form an octamer fusion protein. In some embodiments, fusion proteins comprising a modified knob or hole FC domain may be covalently linked via an R118C mutation and a dimer peptide.

In some embodiments, the present invention provides a nucleic acid molecule comprising a nucleotide sequence encoding a fusion protein of the present invention. In some embodiments, the nucleic acid molecules encoding the fusion proteins of this invention can be part of a recombinant nucleic acid construct comprising any combination of restriction sites and/or functional elements as are well known in the art that facilitate molecular cloning and other recombinant nucleic acid manipulations. Thus, the present invention further provides a recombinant nucleic acid construct comprising a nucleic acid molecule encoding a fusion protein of this invention. The nucleic acid molecule encoding the fusion protein of this invention can be any nucleic acid molecule that functionally encodes the fusion protein of this invention. To functionally encode the fusion protein (i.e., allow the nucleic acids to be expressed), the nucleic acid of this invention can include, for example, expression control sequences, such as an origin of replication, a promoter, an enhancer and necessary information processing sites, such as ribosome binding sites, RNA splice sites, polyadenylation sites and transcriptional terminator sequences.

Non-limiting examples of expression control sequences that can be present in a nucleic acid molecule of this invention include promoters derived from metallothionine genes, actin genes, immunoglobulin genes, CMV, SV40, adenovirus, bovine papilloma virus, etc. A nucleic acid molecule encoding a selected fusion protein can readily be determined based upon the genetic code for the amino acid sequence of the selected polypeptide and/or fragment of interest included in the fusion protein, and many nucleic acids will encode any selected polypeptide and/or fragment. Modifications in the nucleic acid sequence encoding the polypeptide and/or fragment are also contemplated. Modifications that can be useful are modifications to the sequences controlling expression of the polypeptide and/or fragment to make production of the polypeptide and/or fragment inducible or repressible as controlled by the appropriate inducer or repressor. Such methods are standard in the art. The nucleic acid molecule and/or vector of this invention can be generated by means standard in the art, such as by recombinant nucleic acid techniques and/or by synthetic nucleic acid synthesis or in vitro enzymatic synthesis.

The nucleic acids and/or vectors of this invention can be transferred into a host cell (e.g., a prokaryotic or eukaryotic cell) by well-known methods, which vary depending on the type of cell host. For example, calcium chloride transfection is commonly used for prokaryotic cells, whereas calcium phosphate treatment, transduction, cationic lipid treatment and/or electroporation can be used for other cell hosts.

In some embodiments, the present invention provides a DNA construct comprising the nucleic acid molecule comprising a nucleotide sequence encoding a fusion protein of the present invention. In some embodiments, the present invention provides a vector comprising a fusion protein of the present invention. A vector can be any suitable means for delivering a polynucleotide to a cell. In some embodiments, the vector is a plasmid, a viral vector, an expression cassette, a transformed cell, or a nanoparticle.

The present invention further provides compositions. In some embodiments, the present invention provides a pharmaceutical composition comprising a fusion protein, a dimer fusion protein of claim, a tetramer fusion protein, an octamer fusion protein, a nucleic acid molecule, and/or a DNA construct of the present invention, and a pharmaceutically acceptable carrier. By “pharmaceutically acceptable” it is meant a material that is not toxic or otherwise undesirable, i.e., the material may be administered to a subject without causing any undesirable biological effects. In some embodiments, the present invention provides a pharmaceutical composition comprising a fusion protein, a dimer fusion protein of claim, a tetramer fusion protein, a octamer fusion protein, a nucleic acid molecule, and/or a DNA construct of the present invention, a pharmaceutically acceptable carrier, and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and will preferably be in solid or liquid particulate form.

### Methods of Use

An additional aspect of the invention relates to a method of delivering a fusion protein to a subject, comprising administering to the subject a fusion protein, a dimer fusion protein, a tetramer fusion protein, an octamer fusion protein, a nucleic acid molecule, a DNA construct, and/or a pharmaceutical composition of the present invention, thereby delivering the fusion protein to the subject. In some embodiments, the fusion protein may be delivered to the subject via a plasmid, a viral vector, a DNA cassette, a transformed cell, a nanoparticle or any combination thereof.

The viral vector can be an expression vector which contains all of the genetic components required for expression of the nucleic acid in cells into which the vector has been introduced, as are well known in the art. The expression vector can be a commercial expression vector or it can be constructed in the laboratory according to standard molecular biology protocols. The expression vector can comprise viral nucleic acid including, but not limited to, poxvirus, vaccinia virus, adenovirus, retrovirus, alphavirus and/or adeno-associated virus nucleic acid. The nucleic acid or vector of this invention can also be in a liposome or a delivery vehicle, which can be taken up by a cell via receptor-mediated or other type of endocytosis. The nucleic acid molecule of this invention can be in a cell, which can be a cell expressing the nucleic acid whereby a fusion protein of this invention is produced in the cell (e.g., a host cell). In addition, the vector of this invention can be in a cell, which can be a cell expressing the nucleic acid of the vector whereby a fusion protein of this invention is produced in the cell. It is also contemplated that the nucleic acids and/or vectors of this invention can be present in a host organism (e.g., a transgenic organism), which expresses the nucleic acids of this invention and produces the fusion protein of this invention.

In some embodiments, the present invention provides a method of inhibiting IL10 from inducing IL10 signaling in a cell, comprising contacting a fusion protein, a dimer fusion protein, a tetramer fusion protein, an octamer fusion protein, a nucleic acid molecule, a DNA construct, and/or a pharmaceutical composition of the present invention with a substrate comprising the cell and IL10, thereby inhibiting IL10 from inducing IL10 signaling in the cell. In some embodiments, the substrate may be a subject. In some embodiments, the substrate may be an animal subject. In some embodiments, the substrate may be a mammalian subject. In some embodiments, the substrate may be a human or non-human mammal subject. Examples of non-human mammals include but are not limited to rhesus macaques, crab-eating macaques, stump-tailed macaques, pig-tailed macaques, squirrel monkeys, owl monkeys, baboons, chimpanzees, marmosets, spider monkeys, mice, pigs, rabbits, sheep, hamsters, guinea pigs, rats, or ferrets. In some embodiments, the subject may be a human or a rhesus macaque.

In some embodiments, the substrate may be a rhesus macaque subject. In some embodiments, the subject has or is suspected of having an HIV infection, an EBV infection, a CMV infection, malaria, tuberculosis, cancer, or any combination thereof.

In some embodiments, the present invention provides a method of inducing an immune response in a subject, comprising administering to the subject a fusion protein, a dimer fusion protein, a tetramer fusion protein, an octamer fusion protein, a nucleic acid, a DNA, and/or a pharmaceutical composition of the present invention, and an immunogen.

An immunogen useful to the present invention may be any molecule capable of eliciting an immune response in the desired context. For example, a molecule comprising a known or putative antigen or antigenic epitope of an infectious agent, in the context of the desire to elicit an immune response against that infectious agent. In some embodiments, the immunogen may be an SIV immunogen, an HIV immunogen, a cancer immunogen, an EBV immunogen, a CMV immunogen, a tuberculosis immunogen, a malarial immunogen, or any combination thereof.

In some embodiments, the present invention provides a method of priming an immune response to an immunogen that induces an endogenous IL10 response in a subject, comprising: a) administering a fusion protein, a dimer fusion protein, a tetramer fusion protein, an octamer fusion protein, a nucleic acid, a DNA, and/or a pharmaceutical composition of the present invention, to the subject in an amount effective in inhibiting an endogenous IL10 response in the subject; and b) administering the immunogen.

In some embodiments, the present invention provides a method of enhancing an immune response to an immunogen that induces an IL10 response in a subject, comprising administering an effective amount of a fusion protein, a dimer fusion protein, a tetramer fusion protein, an octamer fusion protein, a nucleic acid, a DNA, and/or a pharmaceutical composition of the present invention, and the immunogen to a subject, wherein the immune response is enhanced in the subject as compared to an immune response in a subject to whom the immunogen is administered in the absence of the fusion protein.

In certain embodiments, the synthetic gene, vector, and/or pharmaceutical composition is delivered to the subject, e.g., systemically (e.g., intravenously). In particular embodiments, more than one administration (e.g., two, three, four or more administrations) may be employed to achieve the desired level of gene expression over a period of various intervals, e.g., daily, weekly, monthly, yearly, etc. The most suitable route in any given case will depend on the nature and severity of the condition being treated and on the nature of the particular delivery method that is being used. In embodiments wherein a vector is used, the vector will typically be administered in a liquid formulation by direct injection (e.g., stereotactic injection) to the desired region or tissues. In some embodiments, the vector can be delivered via a reservoir and/or pump. In other embodiments, the vector may be provided by topical application to the desired region or by intra-nasal administration of an aerosol formulation. Administration to the eye or into the ear, may be by topical application of liquid droplets. As a further alternative, the vector may be administered as a solid, slow-release formulation. For example, controlled release of parvovirus and AAV vectors is described by international patent publication WO 01/91803.

Injectables can be prepared in conventional forms, either as liquid solutions or suspensions, solid forms suitable for solution or suspension in liquid prior to injection, or as emulsions. Alternatively, one may administer the vector in a local rather than systemic manner, for example, in a depot or sustained-release formulation. Further, the virus vector can be delivered dried to a surgically implantable matrix such as a bone graft substitute, a suture, a stent, and the like (e.g., as described in U.S. Pat. No. 7,201,898).

Pharmaceutical compositions suitable for oral administration can be presented in discrete units, such as capsules, cachets, lozenges, or tablets, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a vector of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions suitable for parenteral administration can comprise sterile aqueous and non-aqueous injection solutions of the composition of this invention, which preparations are optionally isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit/dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 μg to about 10 grams of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

Pharmaceutical compositions suitable for rectal administration can be presented as unit dose suppositories. These can be prepared by admixing the composition with one or more conventional solid carriers, such as for example, cocoa butter, and then shaping the resulting mixture.

Pharmaceutical compositions of this invention suitable for topical application to the skin can take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharm. Res. 3:318 (1986)) and typically take the form of an optionally buffered aqueous solution of the composition of this invention. Suitable formulations can comprise citrate or bis\tris buffer (pH 6) or ethanol/water and can contain from 0.1 to 0.2M active ingredient.

The delivery methods disclosed herein may be administered to the lungs of a subject by any suitable means, for example, by administering an aerosol suspension of respirable particles comprised of the vectors, which the subject inhales. The respirable particles may be liquid or solid. Aerosols of liquid particles comprising the virus vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the vectors may likewise be produced with any solid particulate medicament aerosol generator, by techniques known in the pharmaceutical art.

Having described the present invention, the same will be explained in greater detail in the following examples, which are included herein for illustration purposes only, and which are not intended to be limiting to the invention.

### EXAMPLES

### Example 1: Design and Function of RhIL10R1-FC

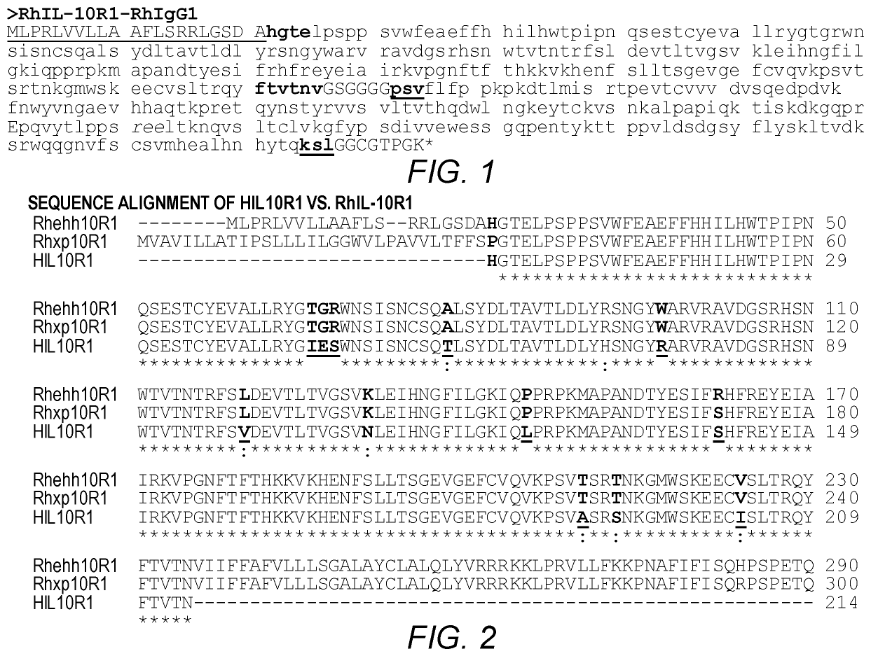

Design of RhIL10R1-FC sequence: The amino acid sequence encoding the RhIL10R1-FC was designed computationally using public sequence databases and structural analysis of the human IL10/IL10R1 complex. The resulting amino sequence of RhIL10R1-FC is shown in FIG. 1. Key components of the protein sequence are highlighted. First, a signal sequence from the IFNγR2 chain (underlined) was used as it was found to increase protein expression relative to wildtype IL10R1 signal sequences. Second, a “GSGGGG” (SEQ ID NO:2) in capital letters is the linker between the RhIL10R1 sequence and the RhIgG1 FC sequence. Finally, the c-terminal capitalized “GGCGTPGK” (SEQ ID NO:3) sequence is the dimer peptide, which allows the FC domains to form a covalent bond between them (see also FIG. 5). The FC of the protein assembles two IL10R1 chains via a disulfide bond that covalently couples two IL10R1-FC chains together. Thus, each RhIL10R1-FC dimer binds one IL10 dimer to form a high affinity protein complex that cannot induce IL10 signaling. DNA encoding the amino acid sequence in FIG. 1 was synthesized by DNA2.0, now called ATUM. For comparative purposes, differences in the amino acid sequences of Rhesus IL10R1 and Human IL10R1 are shown in FIG. 2. As shown in FIG. 2, there are 10 residue differences between Rhesus and human IL10R1.

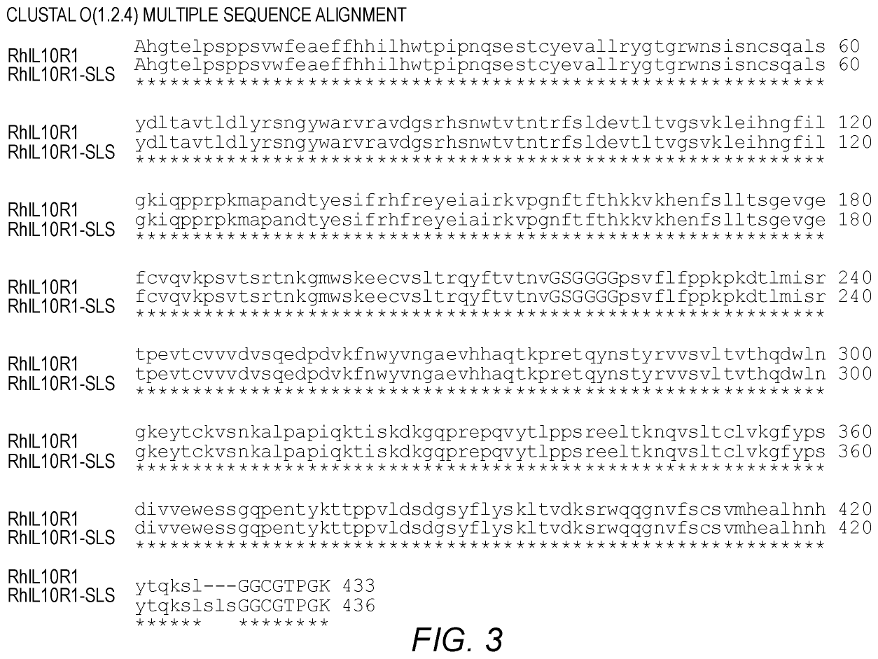

Modification of RhIL10R1-FC to promote dimerization: A cDNA was designed and sent to ATUM (Palo Alto, Calif.) for synthesis (FIG. 1). The cDNA was digested and inserted into the vector pMTA using KpnI and AgeI for expression in insect cells. Initial studies with the protein construct showed that it did not efficiently form disulfide linked dimers, so the sequence (RhIL10R1) was modified by PCR to generate RhIL10R1-SLS (see FIG. 3). The modification added 3 amino acid residues (“SLS”) in the c-terminus of the RhFC. In contrast to the initial protein sequence, the RhIL10R1-FC-SLS sequence efficiently dimerized (FIG. 5). All experiments (FIGS. 5-9) were performed using RhIL10R1-FC-SLS, but was called RhIL10R1-FC.

Cloning of RhIL10R1-FC-SLS: Addition of three amino acid residues (SLS) was performed with the following steps:

### Double Digestion Reaction:

DNA=1 ug, 10×NEB 1.1 buffer=5 ul, KpnI=1 ul, AgeI=1 ul
Sterile water to make up volume=50 ul
The reaction was carried out at 37° C. for 2 hr. After the reaction was complete, both the double digested vector and insert were run on 1% agarose gel. Vector and insert pieces were cut out from the gel using clean and sharp scalpel. The DNA from the gel was purified using QIAGEN gel extraction kit (cat #28704). Purified double digested vector and insert were ligated using T4 DNA ligase (NEB cat #M0202).

### Ligation Reaction:

10×T4 Ligase buffer=2 ul

### Vector DNA=50 ng

### Insert DNA=38 ng

Sterile water to make up 20 ul

### T4 Ligase=1 ul

The ligation reaction was incubated at room temperature for 30 minutes. To transform, 5 ul of the reaction mix was added to 50 uL of XL1 Blue Super competent cells, incubated on ice for 10 minutes and plated on Luria agar+100 ug/ml Carbenicillin plates. The plate was incubated at 37° C. overnight. Three single colonies were picked to grow up mini preparations (10 ml LB 100 ug/ml Carbenicillin). The next day, the DNA was purified using QIAGEN Miniperp kit (Cat #27106) and sequenced. A clone containing the additional sequence was grown for Maxi prep (100 ml LB+100 ug/ml Carbenicillin).

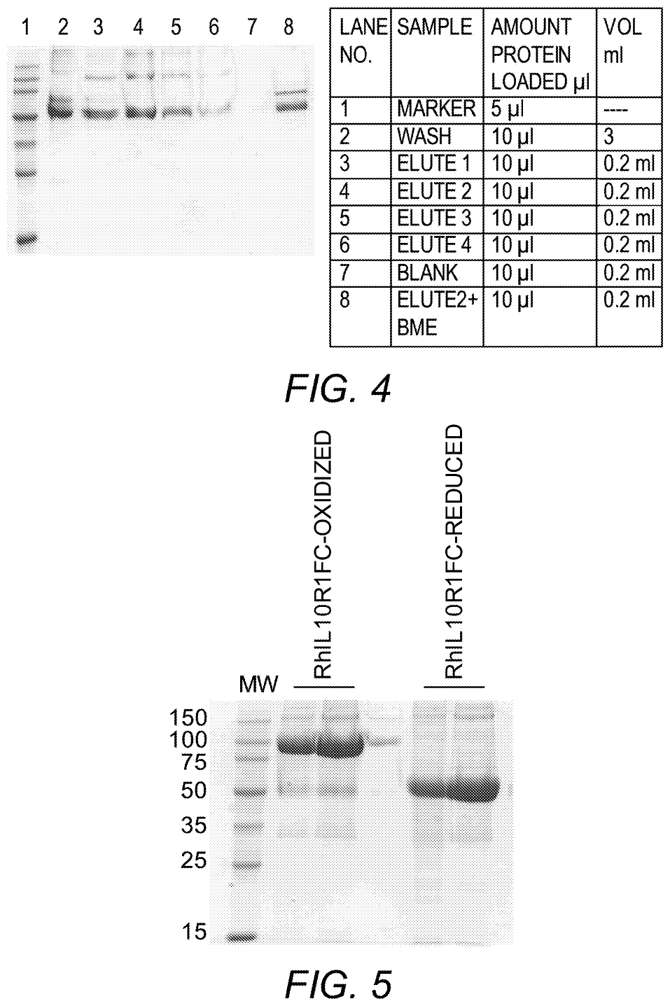

Large scale DNA was purified with a QIAGEN Maxi prep Kit (Cat #12262), and used for transfection into S2 Drosophila cells. For transient expression, a 10 ml culture was induced using 0.5 mM Cu₂SO₄ and the culture was grown for five days after induction and harvested. Harvested protein was purified using 50 ul bed volume of protein A resin to check if the pMTA/Rh-IL10R1Fc was expressed as homodimer. The pMTA/RhIL10R1FC did not form homodimers but rather the protein was expressed as a monomer (FIG. 4). Therefore, the SLS amino acid sequence was added to the FC region of the protein using overlapping primers, as described below.

Rh-Fc-sls-Forward:
(SEQ ID NO: 44)
agt ctg agt gga ggt tgc gga aca ccg ggt aaa tga
tga acc ggt
Rh-Fc-sls-Reverse:
(SEQ ID NO: 45)
act cag act gag gct ctt ctg ggt gta gtg gtt gtg

PCR Reaction: (with HOT Start)
Sterile water=38.5 ul
10×Pfu or Taq buffer=5 ul
Rh-Fc-sls-For=1.5 ul (10 nM stock)
Rh-Fc-sls-Rev=1.5 ul (10 nM stock)

### Template DNA (Rh-IL10R1Fc)=1 ul (10 pg)

dNTP=1.5 ul (100 nM)
Pfu polymerase or TaqPol=1 ul

### Program Used to Generate Mutation

### Step 1=95° for 4 min

### Step 2=95° for 0:30 min

### Step 3=55° for 0:30 min

### Step 4=97° for 1: min

Step 5=Go to Step 2 for 35 times

### Step 6=72° for 10 min

Step 7=4° forever

After the PCR was run, the DNA was digested with DPN I. The DPNI reaction was for 1 hr at 37° C. The enzyme activity was disrupted by incubating at 80° C. for 1.5 min. The mixture was transformed into XL-1 super competent cells, followed by plating. The next morning 3 single colonies were picked to grow up mini preps (10 ml Luria broth+100 ug/ml Carbenicillin).

### DPN I Digestion Reaction

Sterile water=25 ul
Cut smart buffer (NEB)=3 ul

### DPN I=1 ul

The following day DNA was purified using QIAGEN Miniprep kit (Cat #27106) and sequenced. Clones containing the correct mutation were grown for a Maxi prep (100 ml Luria broth+100 ug/ml Carbenicillin). Large scale DNA was purified using the QIAGEN Maxi prep Kit (Cat #12262), and used for transfection into S2 Drosophila cells. To generate stable lines, the plasmid was co-transfected with pCoHygro resistance plasmid. Transfected cells were selected using Hygromycin B (Calbiochem Cat #400052) for 19 days and then expanded and induced by adding 0.5 mM Cu¬2SO4 in serum free (Lonza cat #12-730Q).

Large-scale Protein Expression: RhIL10R1-FC was expressed by transfecting the expression plasmid into Drosophila S2 cells using manufacture's protocols (Invitrogen). Stable cell lines were selected with hygromycin and protein expression was induced by addition of 0.5 mM Cu₂SO₄ at a cell density of 5×10⁶ cells ml⁻¹, in serum free media (Lonza) containing 20 mM L-glutamine. Expressed media was harvested after 7 days, clarified by centrifugation, and filtered through a 0.2 μm PES filter. RhIL10R1-FC was purified using a 1 mL Protein A affinity column (Thermo scientific) equilibrated in 20 mM Tris HCl pH 8.0, 150 mM NaCl. Bound protein was washed with 50 column volumes of equilibration buffer and eluted in 2M Arginine pH 3.8. The pH of the column fractions was adjusted by addition of 1M Tris HCl pH 8.0, followed by dialysis against 20 mM Tris HCl pH 8.0, 150 mM NaCl. Pooled fractions were concentrated in a centricon concentrator for further analysis.

Neutralization Assays: RhIL10/RhIL10R1-FC and RhIL10/IL10 MAb mixtures at various concentrations (protein/cell mixtures 200 uL total volume) were incubated in 96 well plates for 30 minutes at 37 degrees C. and then added to Rhesus PBMCs (4×10⁵ cells per well) which were then stimulated with 5 μg/mL LPS, followed by incubation for 20-24 hours. Following the ˜24 hr incubation the supernatants were collected and 100 μL was assayed for Rhesus IL10 levels and 100 μL was assayed for Rhesus IL-12 levels by ELISA (Ucytech) using the manufacturer's protocols.

Surface Plasmon Resonance (SPR): SPR data collection and analysis was performed using a Biacore T-200 (GE healthcare). RhIL10R1-FC and Rh antibody were coupled to CMS Biacore chips using an anti-human FC coupling kit, according to the manufacturer's instructions (GE healthcare). IL10s were injected over the surfaces at 100 μL/min in a running buffer of 10 mM HEPES, 150 mM NaCl, 0.015% P20 (GE Healthcare), and 150 μg/mL bovine serum albumin (BSA). Fresh protein surfaces were prepared for each cycle by a 3 minute injection of 10 mM glycine, pH 1.7, followed by an injection of new RhIL10R1-FC or antibody. Sensorgrams were fit to appropriate models using Biacore T-200 evaluation software version 1.0.

Expression and purification of RhIL10R1-FC: The cDNA encoding RhIL10R1-FC was cloned into a pMTA vector for expression in insect cells. Protein expression was induced by CuSO4 resulting in the protein being secreted into the media. Following expression, RhIL10R1-FC was purified by protein A chromatography (FIG. 5). The calculated molecular weight of the protein is 51,026. Thus, the RhIL10R1-FC covalent dimer would be expected to run at a molecular weight of ˜102,000 under oxidizing conditions. As shown in FIG. 5, RhIL10R1-FC forms a covalent dimer under oxidizing conditions. Upon placing the protein into reducing conditions, the disulfide bond is broken and the protein runs as a monomer (FIG. 5).

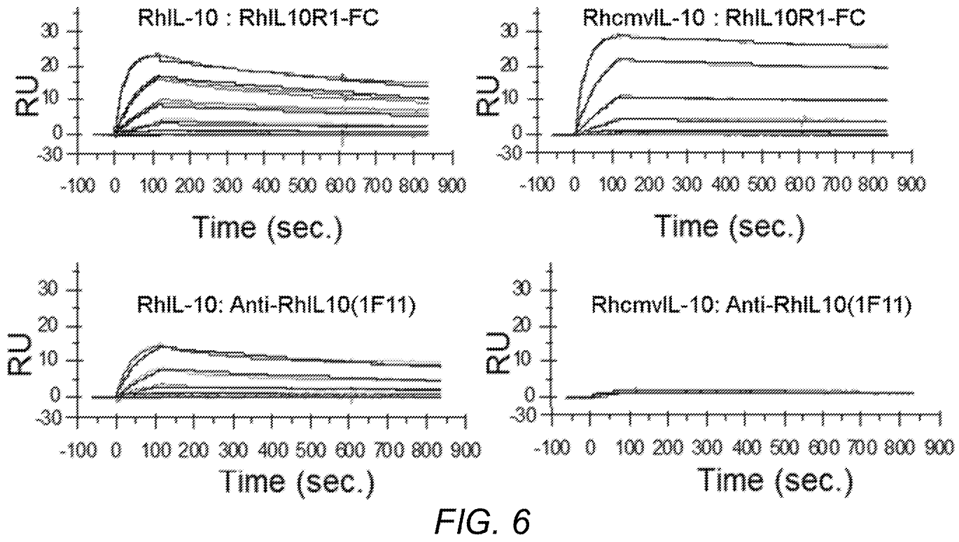

Kinetic analysis of IL10 binding by RhIL10R1-FC and Ab 1F11: The binding kinetics of IL10/RhIL10R1-FC, and the IL10/anti-RhIL10 antibody (1F11) interactions were determined using surface plasmon resonance (SPR). The sensorgrams for RhIL10R1-FC and 1F11 binding to RhIL10 and RhCMVIL10 are shown in FIG. 6. The SPR sensorgrams demonstrate that 1F11 does not bind to the RhCMVIL10, whereas RhIL10R1-FC binds to cellular RhIL10 and RhCMVIL10. The binding constants for each protein-protein interaction are reported in Table 3. In Table 3, ka units are M⁻¹sec⁻¹, kd units are sec⁻¹, RU max is calculated dividing the molecular weight (MW) of the analyte by the MW of the ligand. This fraction is multiplied by the amount of ligand coupled to the chip surface to yield RUmax. For example, (MW^{analyte}/MW^{ligand})*RUs of ligand coupled to chip surface. Surface activity is calculated as RUobs/RUmax*100.

As shown in Table 3, there is residual binding to RhCMVIL10 by 1F11. However, 1F11 only binds 14% of the expected amount of RhCMVIL10 and its affinity for RhCMVIL10 (KD=7.6 nM) is 104-fold weaker than RhIL10R1-FC. In addition to the potent binding of RhIL10R1-FC to RhCMVIL10 (KD=72 pM), the affinity of RhIL10R1-FC for cellular RhIL10 (KD=136 pM) is 4.8-fold greater than 1F11. In summary, RhIL10R1-FC binds to both RhCMVIL10 and RhIL10 with greater affinity than 1F11 obtained from the NIH Nonhuman Primate Reagent Resource

Functional Analysis of RhIL10R1-FC: Rhesus monocytes stimulated with lipopolysaccharide (LPS) produce the inflammatory cytokine IL12. LPS-induced IL12 production by monocytes is inhibited by IL10. Thus, the assay can be used to characterize the ability of RhIL10R1-FC, or Ab 1F11, to neutralize IL10 bioactivity, where neutralization of IL10 in the assay results in high IL12 levels.

For the assay, RhIL10, or RhCMVIL10, are incubated with RhIL10R1-FC, or 1F11, for 30 minutes at 37° C. and then added to 400,000 Rhesus PBMCs (200 μL), followed by stimulation of the cells with 5 μg/mL LPS for 24 hrs. After 24 hrs, the cell supernatants were collected and RhIL12 levels were measured by ELISA (Ucytech). The data were normalized to the level of RhIL12 produced in the absence of IL10. Thus, normalized values greater than or equal to one suggest complete neutralization of IL10 biological activity. For example, the restoration of RhIL12 to levels observed in the absence of IL10.

Cellular RhIL10 Neutralization: The first assay compared the ability of RhIL10R1-FC and Ab 1F11 to neutralize RhIL10 (FIG. 7). The plot shows that RhIL10R1-FC blocks almost all RhIL10-induced RhIL12 production at a concentration of −50 ng/mL, which corresponds to a concentration of 490 pM. In addition, calculations in Table 4, based on FIG. 7, demonstrate the molar stoichiometry of RhIL10R1-FC required to neutralize RhIL10. In Table 4, concentrations of RhIL10R1-FC in the top row, in ng/mL, were used in FIG. 7 to neutralize RhIL10 activity. The calculations in the table convert these values to molar concentrations (M) and moles of RhIL10R1-FC required to neutralize RhIL10 (constant 10 ng/mL in each well).

From these calculations, the molar stoichiometry of RhIL10R1-FC required to neutralize RhIL10 is approximately two. This implies that 2 molecules of RhIL10R1-FC are sufficient to neutralize one molecule of RhIL10. In contrast, Ab 1F11 requires 6000-12,000 Ab molecules to neutralize 1 molecule of RhIL10 (Table 5), and the Ab only completely restores RhIL12 levels in 2 of the 6 animals (FIG. 7). In Table 5, the top concentrations of Ab 1F11 are in ng/mL and were used in FIG. 7 to neutralize RhIL10 activity. The calculations in Table 5 convert these values to molar concentrations and moles of 1F11 Ab required to neutralize RhIL10 (constant 10 ng/mL in each well). These data suggest RhIL10 neutralization studies performed in rhesus macaques with Ab 1F11 are likely to provide incorrect data on the role of IL10 in shaping the immune response.

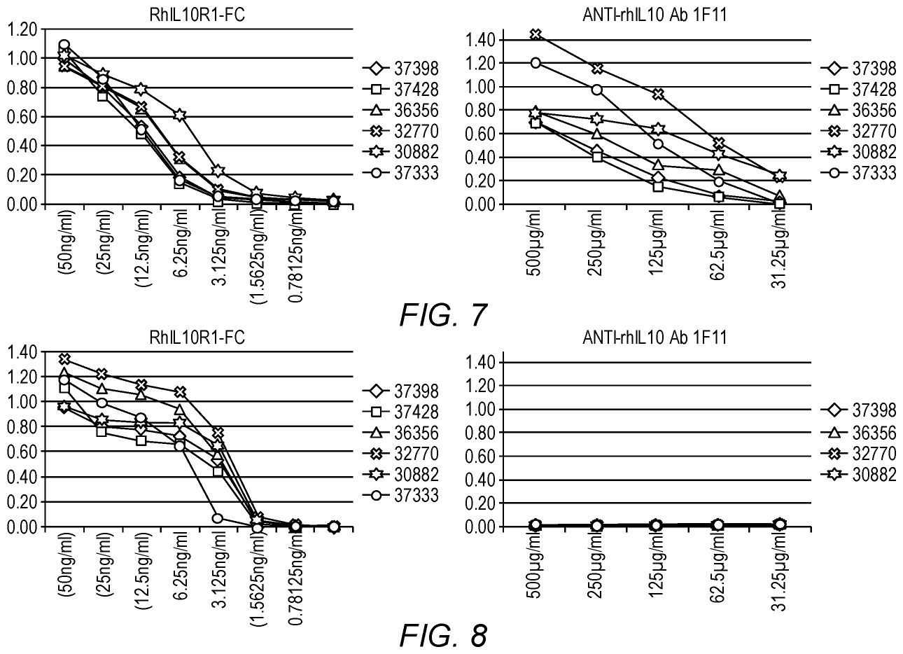

RhCMVIL10 Neutralization: The ability of RhIL10R1-FC and Ab 1F11 to neutralize RhCMVIL10 was also compared in the same assay format (FIG. 8). Consistent with the SPR studies, RhIL10R1-FC potently neutralized RhCMVIL10 biological activity, while Ab 1F11 could not neutralize RhCMVIL10 at any concentration tested. The concentrations of RhIL10R1-FC required for RhCMVIL10 neutralization were almost identical to concentrations necessary for cellular RhIL10 (˜490 pM).

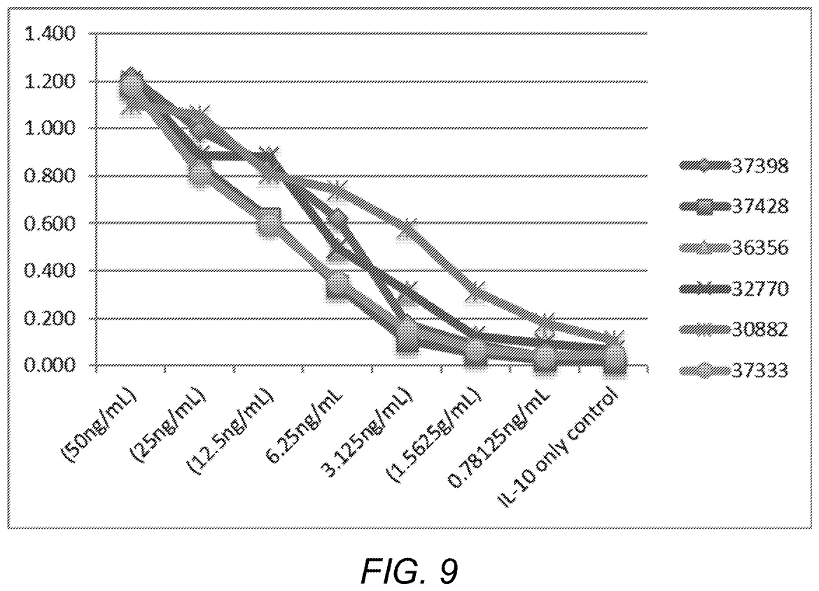

Human cellular IL10 Neutralization: The ability of RhIL10R1-FC to neutralize human cellular IL10 was also evaluated (FIG. 9). RhIL10R1-FC potently neutralized human cellular IL10 mediated suppression of RhIL12. The concentrations of RhIL10R1-FC required for neutralization of human cellular IL10 were almost identical to concentrations necessary for cellular RhIL10 (˜490 pM). Therefore, RhIL10R1-FC provides a potent antagonist of IL10 biological activity, and could be given prior to, and/or during vaccinations.

### Example 2: Design and Function of Multimeric EVGBP Fusion Proteins

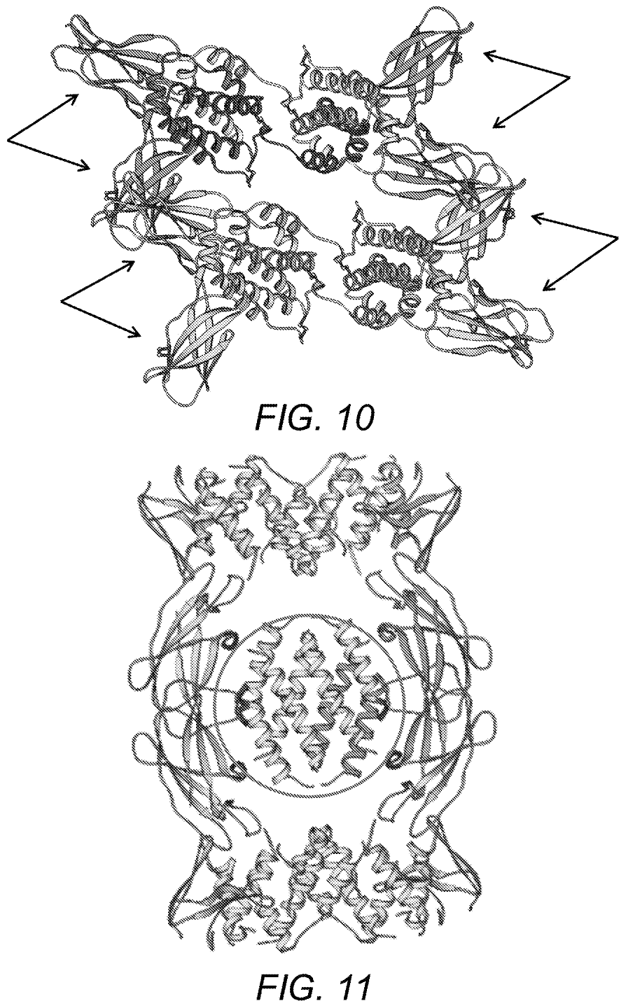

The IL10R1-FC protein described above provides one example of how IL10R1 can be assembled to efficiently neutralize cellular and viral IL10 biological activity. However, the crystal structure of the IL10-IL10R1 complex demonstrated that IL10 and IL10R1 receptors can make higher order complexes, containing two IL10s and four IL10R1s (FIG. 10). This concept allows for additional novel strategies to assemble IL10R1 multimers that form higher affinity interactions with IL10 and potently neutralize cellular and viral IL10s.

The crystal structure of ectromelia virus interferon-γ binding protein (EVGBP, also called the IFNγ binding protein) shows that the molecule is a tetramer. EVGBP forms a tetramer using a novel tetramerization domain (D3), which is a 53 amino acid peptide that does not share sequence identify with any other protein in the human proteome. FIG. 11 demonstrates that the D3 domain can form tetramers in the absence of the IFNγ binding components of EVGBP.

A method for controlling IL10R1 multimer formation using the D3 domain: The putative D3 domains from 92 IFNγBP sequences from the poxvirus bioinformatic resource were aligned. A representative number of sequences from that alignment are shown in FIG. 12, showing similarities and differences relative to EVGBP D3.

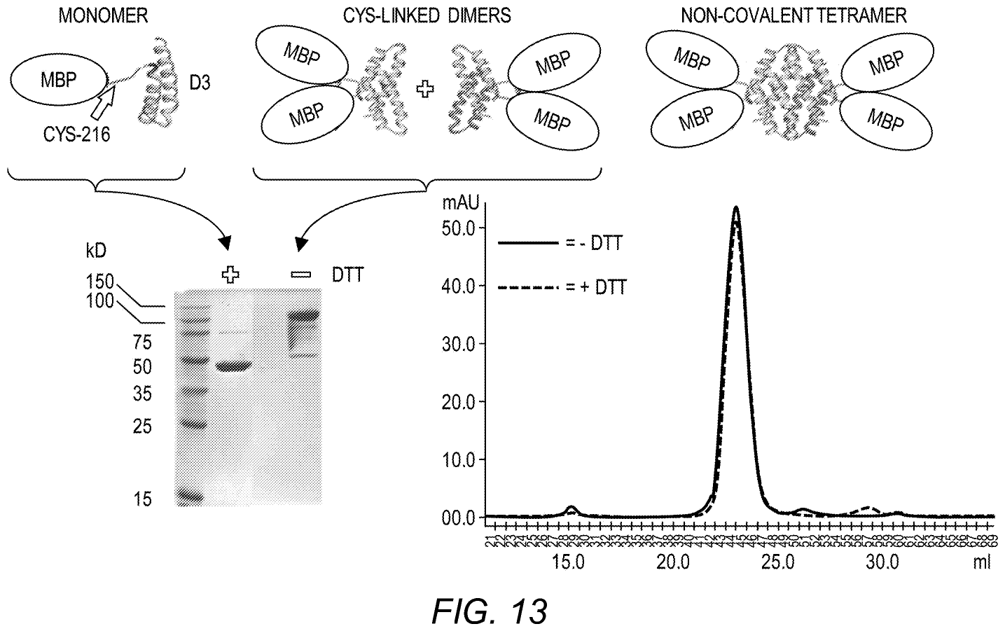

EVGBP D3 forms tetramers and does not require Cys 216 for tetramer formation: The EVGBP D3 peptide contains a cysteine residue at its N-terminus, which may be required to form a stable tetramer. To determine if Cys216 is important for tetramerization, the D3 peptide was expressed as a maltose binding protein (MBP) D3 peptide fusion as shown in FIG. 13. The protein was expressed and run on a gel filtration column with, or without, the reducing agent DTT. The chromatogram demonstrates that the MBP-D3 fusion runs as a tetramer regardless of reducing agent addition. When the protein is run on a SDS-PAGE gel, addition of DTT leads to MBP-D3 monomer formation. In the absence of DTT, MBP-D3 runs as a cysteine-linked dimer (via a cys-216 cys-216 disulfide bond).

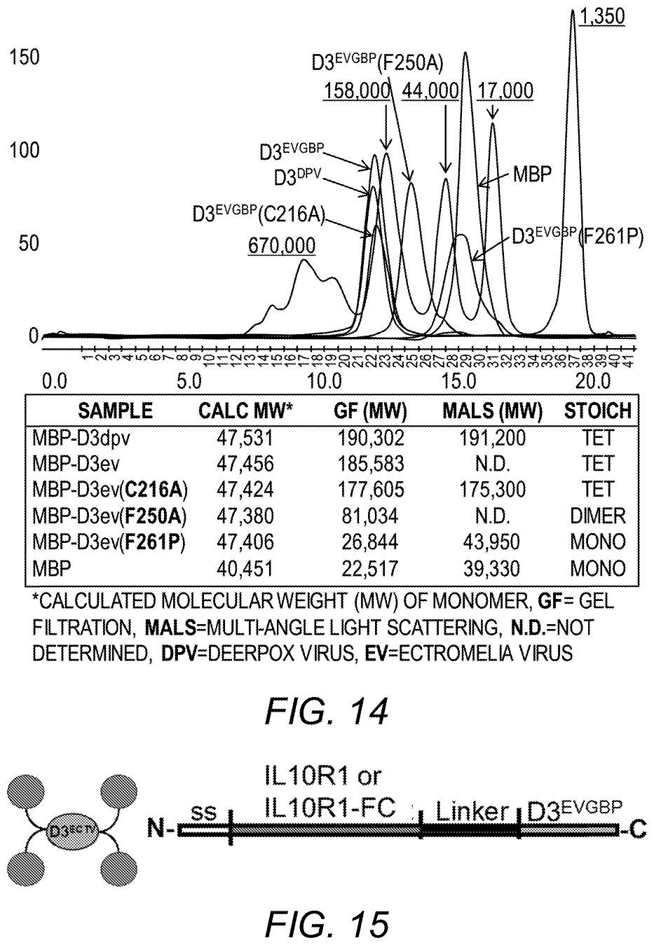

Characterization of EVGBP D3 mutants that control D3 assembly into monomers, dimers, and tetramers: Based on the results of FIG. 13, a series of structure-based alanine mutants of the EVGBP D3 were made in the MBP-D3 fusion protein to further test D3 peptide assembly. First, to confirm the results shown in FIG. 13, a cys-216-ala mutation was made (D3 C216A). As previously shown using reducing agent, the C216A mutant forms a tetramer like the “wildtype” D3 peptide (FIG. 14). The MBP-D3 fusion could be converted to a dimer by expressing the EVGBP F250A mutant and to a monomer with a F261P mutation. Thus, mutations in EVGBP D3 domain were identified to convert the peptide sequence into a tetramer, dimer, and monomer. To validate other D3 peptides that exhibit sequence diversity, relative to EVGBP, the DPV-848 sequence underlined in FIG. 12 was expressed as a MBP fusion protein and characterized for its ability to assemble tetramers. As shown in FIG. 14, the MBP-D3DPV ran on the gel filtration column at the same location as the EVGBP D3 protein. In addition to using molecular weight standards in the gel filtration runs, the molecular weights of the fusion proteins were also evaluate using multi-angle light scattering (MALS). The MALS data are consistent with the molecular weights assigned using a calibrated gel filtration column. This suggests that all EVGBP D3 sequences form tetramers and are available to optimize for the creation of multimeric IL10R1 proteins to neutralize the biological activities of human and viral IL10.

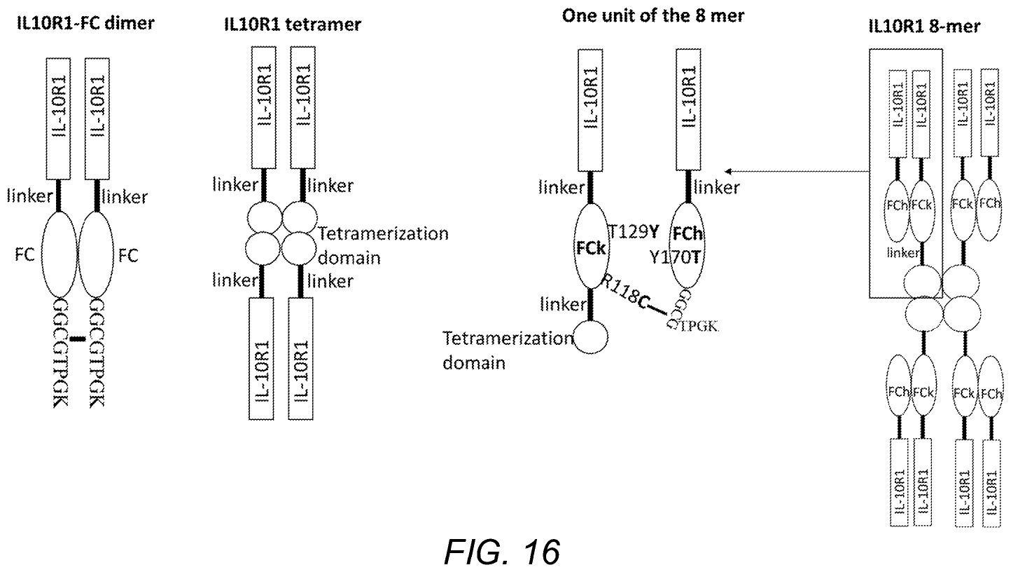

Strategies for combining IL10R1 and EVGBP D3 technologies: The combination of the IL10R1-FC and the D3 assembly domains provide novel strategies to assemble IL10R1 multimers to neutralize IL10 biological activity. As shown in FIG. 15, the D3 domain provides a strategy to assemble four IL10R1s that can form a high affinity complex between IL10 and IL10R1 as shown in the IL10-IL10R1 crystal structure (FIG. 10). In addition, using the IL10R1-FC and D3-EVGBP in combination provides a way to assemble 8 IL10R1s (FIG. 16). To form the IL10R1 8mers, a knob and hole strategy for the FC would be used as previously described, with the modifications of the FC domain as shown in FIG. 17. In some embodiments, a coiled coil domain can be used to assemble the IL10R1 chains.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

TABLE 1
Amino Acids

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 2
Modified Amino Acids

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | aIle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

TABLE 3
SPR binding parameters for RhIL10R1-FC and Ab 1F11

| Analyte | Ligand | k a | k d | K D | RU max | RU obs | surface activity |
|---|---|---|---|---|---|---|---|
| RhCMVIL10 | RhIL10R1-FC | 2.35E+06 | 1.70E−04 | 7.24E−11 | 29.00 | 29.00 | 100% |
| RhIL-10 | RhIL10R1-FC | 3.35E+06 | 4.54E−04 | 1.36E−10 | 19.10 | 19.50 | 100% |
| RhCMVIL10 | Ab 1F11 | 8.44E+04 | 6.38E−04 | 7.56E−09 | 14.00 | 2.00 | 14% |
| RhIL-10 | Ab 1F11 | 1.04E+06 | 5.90E−04 | 5.67E−10 | 14.20 | 15.00 | 100% |

TABLE 4
Concentrations and stoichiometry of RhIL10R1-FC required for neutralization in FIG. 7 assay

| | | | | | | | |
|---|---|---|---|---|---|---|---|
| RhIL10R1-FC (ng/mL) | 5.00E−08 | 2.50E−08 | 1.25E−08 | 6.25E−09 | 3.13E−09 | 1.56E−09 | 7.81E−10 |
| RhIL10R1-FC (M) | 4.90E−10 | 2.45E−10 | 1.23E−10 | 6.13E−11 | 3.06E−11 | 1.53E−11 | 7.66E−12 |
| moles RhIL10 | 5.35E−14 | 5.35E−14 | 5.35E−14 | 5.35E−14 | 5.35E−14 | 5.35E−14 | 5.35E−14 |
| moles Rh10R1-FC | 9.80E−14 | 4.90E−14 | 2.45E−14 | 1.23E−14 | 6.13E−15 | 3.06E−15 | 1.53E−15 |
| Stoichiometry | 1.83 | 0.92 | 0.46 | 0.23 | 0.11 | 0.06 | 0.03 |
| RhIL10R1-FC/RhIL10 | | | | | | | |

TABLE 5
Concentrations and stoichiometry of Ab 1F11
required for neutralization in FIG. 7 assay

| | | | | | |
|---|---|---|---|---|---|
| Ab 1F11 | 5.00E−04 | 2.50E−04 | 1.25E−04 | 6.25E−05 | 3.13E−05 |
| Ab conc. (M) | 3.33E−06 | 1.67E−06 | 8.33E−07 | 4.17E−07 | 2.08E−07 |
| moles RhIL10 | 5.35E−14 | 5.35E−14 | 5.35E−14 | 5.35E−14 | 5.35E−14 |
| moles 1F11 | 6.66667E−10 | 3.3333E−10 | 1.667E−10 | 8.33E−11 | 4.17E−11 |
| Stoichiometry | 12458.67 | 6229.33 | 3114.67 | 1557.33 | 778.67 |
| 1F11/RhIL10 | | | | | |

1. A fusion protein comprising:
a) an IL10R1 domain;
b) a linker peptide; and
c) a multimerization domain.

2. The fusion protein of claim 1, further comprising a heterologous signal peptide.

3. The fusion protein of claim 2, wherein the heterologous signal peptide comprises the signal peptide of IFNγR2, the amino acid sequence MLPRLVVLLAAFLSRRLGSDA (SEQ ID NO:1), or any combination thereof.

4. The fusion protein of claim 1, wherein the linker peptide comprises the amino acid sequence GSGGGG (SEQ ID NO:2).

5. The fusion protein of claim 1, wherein the multimerization domain comprises an FC domain of an IgG molecule.

6. The fusion protein of claim 5, wherein the FC domain is the IgG1 domain of a human, or a non-human mammal.

7. The fusion protein of claim 5, wherein the FC domain is the IgG1 domain of a rhesus macaque.

8. The fusion protein of claim 5, wherein the FC domain is the IgG domain of IgG1, IgG2, IgG3, IgG4, modified variants thereof or active fragments thereof.

9. The fusion protein of claim 5, wherein the multimerization domain further comprises a dimer peptide.

10. The fusion protein of claim 9, wherein the dimer peptide comprises the amino acid sequence GGCGTPGK (SEQ ID NO:3).

11. The fusion protein of claim 9, wherein the FC domain is a modified FC domain with a Y170T mutation.

12. The fusion protein of claim 2, wherein the multimerization domain comprises an IFNγ binding protein, modified variants thereof or active fragments thereof.

13. The fusion protein of claim 12, wherein the IFNγ binding protein, modified variant thereof, or active fragment thereof, is an IFNγ binding protein from a poxvirus.

14. The fusion protein of claim 12, wherein the IFNγ binding protein, modified variant thereof, or active fragment thereof, is an IFNγ binding protein from ectromelia virus (EV), myxoma virus (MYXV), or deerpox virus (DPV).

15. The fusion protein of claim 12, wherein the IFNγ binding protein, modified variant thereof, or active fragment thereof, is EVGBP, D3-EVGBP, MYXV-LAU_171, DPV-W949_93-011, or any combination thereof.

16. The fusion protein of claim 1, wherein the IL10R1 domain is an IL10R1 domain of a human or a non-human mammal.

17. The fusion protein of claim 1, wherein the IL10R1 domain is an IL10R1 domain of a rhesus macaque.

18. (canceled)

19. The fusion protein of claim 18, wherein the FC domain is a modified FC domain with T129Y and R118C mutations.

20. The fusion protein of claim 12, wherein the IFNγ binding protein, modified variant thereof, or active fragment thereof, is a modified D3-EVGBP with an F261P mutation.

21. The fusion protein of claim 12, wherein the IFNγ binding protein, modified variant thereof, or active fragment thereof, is a modified D3-EVGBP with an F250A mutation.

22. A fusion protein comprising the amino acid sequence:
(SEQ ID NO: 4)
MLPRLVVLLAAFLSRRLGSDAHGTELPSPPSVWFEAEFFHHILHWTPI
PNQSESTCYEVALLRYGTGRWNSISNCSQALSYDLTAVTLDLYRSNGY
WARVRAVDGSRHSNWTVTNTRFSLDEVTLTVGSVKLEIHNGFILGKIQ
PPRPKMAPANDTYESIFRHFREYEIAIRKVPGNFTFTHKKVKHENFSL
LTSGEVGEFCVQVKPSVTSRTNKGMWSKEECVSLTRQYFTVTNVGSGG
GGPSVFLFPPKPKDTLMISRTPEVTCVVVDVSQEDPDVKFNWYVNGAE
VHHAQTKPRETQYNSTYRVVSVLTVTHQDWLNGKEYTCKVSNKALPAP
IQKTISKDKGQPREPQVYTLPPSREELTKNQVSLTCLVKGFYPSDIVV
EWESSGQPENTYKTTPPVLDSDGSYFLYSKLTVDKSRQQQGNVFSCSV
MHEALHNHYTQKSLGGCGTPGK*.

23. A fusion protein comprising the amino acid sequence:
(SEQ ID NO: 5)
MLPRLVVLLAAFLSRRLGSDAHGTELPSPPSVWFEAEFFHHILHWTPI
PNQSESTCYEVALLRYGIESWNSISNCSQTLSYDLTAVTLDLYHSNGY
RARVRAVDGSRHSNWTVTNTRFSVDEVTLTVGSVNLEIHNGFILGKIQ
LPRPKMAPANDTYESIFSHFREYEIAIRKVPGNFTFTHKKVKHENFSL
LTSGEVGEFCVQVKPSVASRSNKGMWSKEECISLTRQYFTVTNVGSGG
GGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSV
MHEALHNHYTQKSLSLSGGCGTPGK*.

24-35. (canceled)

36. A method of delivering a fusion protein to a subject, comprising administering to the subject the fusion protein of claim 1, thereby delivering the fusion protein to the subject.

37. (canceled)

38. A method of inhibiting IL10 from inducing IL10 signaling in a cell, comprising contacting the fusion protein of claim 1, with a substrate comprising the cell and IL10, thereby inhibiting IL10 from inducing IL10 signaling in the cell.

39-40. (canceled)

41. A method of inducing an immune response in a subject, comprising administering to the subject the fusion protein of claim 1, and an immunogen.

42-46. (canceled)
